SEQUENCE LISTING (3 sequences)

SEQ ID NO: 1 - DNA, 1396 bp, Homo sapiens
agccaccatcgaggaaggggcatgtgctggacgcggacacatgatccgagggaccctgctgggtggaactaagaaagtccagcagactgtgcatgctcctgtccccactcacaggcccacgcagcgaggggggcccctcttctgtgtgcgtctggaaggtcgctgcccagggaggaaatgcctttaaccggcgtggggccgggcagctgcaggaggcagatcatccgggctctgtgcctcttgctacttctcctccacgccggctctgccaagaatatctggaaacgggcattgcctgcgaggctggccgagaaatcccgtgccgaggaggcgggcgcgcccggcggcccccggcagccccgagcagaccgctgcccgccgcctccgcggacgctgccccccggcgcctgccaggccgcgcgctgtcaggcggactccgagtgcccgcggcaccggcgctgctgctacaacggatgcgcctacgcctgcctagaagctgtgccgcccccgccagtcttagactggctggtgcagccgaaacctcgatggcttggtggcaatggctggctcctggatggccctgaggaggtgttacaagcagaggcgtgcagcaccacggaggatggggccgaacccctgctctgtccctcgggctatgagtgccacatcctgagcccaggtgacgtggccgaaggtatccccaaccgtgggcagtgcgtcaagcagcgccggcaagcagatgggcgaatcctacgacacaaactttacaaagaatatccagaaggtgactcaaagaatgtggcagaacctggaaggggacaacagaagcactttcagtaaagcaacggcaagcagctaggttgcaagaacattcctctactttctgctaagccttggaaacagttgggaaaagtagtttgaccctcacagttcacattcagctcagcagagcaagaccccagagatgcttagagacaggacacctggccctcaaacccagtttggcccagcctggttgggtgactttgtgggagccacttaacagctctgggtccctgttttaccatcctgggagcaaggccctgcagctccacgagacctttaccccgggaagaagccgccgcccatgaaagcatttctgaagcccctttctaagacaaggctcagcatcttgatatttttgacagattcctcccaagtctggctctgggaggtatgtacccatctcaaatgttcccaagataaattcatccttcaggaaatggaaatgaacttgcttactaatgtgtgattcctagttgtagccaccggatgtgctgaggcctaaatgttagcaggtgggaggaggccacagaacaataaaaacaaccaaataagaaaaaaaaaaaaaaaaaaa

SEQ ID NO: 2 - protein, 220 aa, Homo sapiens
MPLTGVGPGSCRRQIIRALCLLLLLLHAGSAKNIWKRALPARLAEKSRAEEAGAPGGPRQPRADRCPPPPRTLPPGACQAARCQADSECPRHRRCCYNGCAYACLEAVPPPPVLDWLVQPKPRWLGGNGWLLDGPEEVLQAEACSTTEDGAEPLLCPSGYECHILSPGDVAEGIPNRGQCVKQRRQADGRILRHKLYKEYPEGDSKNVAEPGRGQQKHFQ

SEQ ID NO: 3 - protein, 220 aa, Homo sapiens [sequence omitted]

(19) United States
(12) Patent Application Publication
(10) Pub. No.: US 2010/0284968 A1
(43) Pub. Date: Nov. 11, 2010

(54) Use of PS20/WFDC1 and Interferons to Diagnose, Monitor and Treat Viral Diseases

(76) Inventors: Eleanor Fish, Toronto (CA); Ehtesham Baig, Markham (CA); Annapuma Vyakamam, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/738,121

(22) PCT Filed: Oct. 15, 2008
(86) PCT No.: PCT/CA08/01800

§ 371 Date: Jul. 14, 2010

Related U.S. Application Data
(60) Provisional application No. 60/998,937, filed on Oct. 15, 2007.

(51) Int. Cl. A61K 38/21; C12Q 1/70; A61P 31/12

(52) U.S. Cl. 424/85.4; 435/5

(57) ABSTRACT

Methods for detecting, diagnosing or monitoring viral diseases in a subject are described comprising measuring ps20 polypeptides or ps20 polynucleotides and interferons and interferon polynucleotides in a sample from the subject. The invention also contemplates therapeutic applications for viral diseases employing modulators of ps20 polypeptides and ps20 polynucleotides in combination with interferon or interferon polynucleotides.

### FIELD OF THE INVENTION

The invention relates to compositions and methods for the diagnosis, treatment, prevention and amelioration of viral diseases.

### BACKGROUND OF THE INVENTION

Considerable attention has been drawn at a global level to the serious threat to humans by new, emerging and re-emerging viral diseases. There is an overwhelming need for effective antiviral therapeutics, particularly broad spectrum antivirals. One approach to antiviral drug design is to target viral proteins, or parts of proteins, that can be disabled. Targets are preferably selected that are common across many strains of a virus, or among different species of viruses, so a single drug will have broad effectiveness. Another approach to antiviral drug design is to interfere with the ability of a virus to infiltrate a target cell. Examples of entry-blockers include amantadine and rimantadine which have been introduced to combat influenza, and pleconaril which works against rhinoviruses. A third approach is to target the processes that synthesize virus components after invasion. Nucleotide or nucleoside analogues such as acyclovir and zidovudine (AZT) have been developed that deactivate enzymes that synthesize viral RNA or DNA once the analogue is incorporated. Other antiviral strategies have been based on RNaseH, integrase, mRNA and ribozymes.

### SUMMARY OF THE INVENTION

The invention relates to methods for the diagnosis and therapy of viral diseases.

In an aspect, the invention relates to a method for the diagnosis of a viral disease comprising detecting one or more ps20 polypeptides and/or ps20 polynucleotides, and one or more interferons and/or polynucleotides encoding interferons.

In an embodiment of the invention, a method is provided for diagnosing a viral disease in a subject comprising:
(a) detecting in a sample from a subject ps20 polypeptides or ps20 polynucleotides and interferons or polynucleotides encoding interferons; and
(b) comparing the detected amounts with amounts detected for a standard.

The term “detect” or “detecting” includes assaying or otherwise establishing the presence or absence of the target ps20 polypeptides, ps20 polynucleotides, and interferons, polynucleotides encoding interferons, subunits thereof, or combinations of reagent bound targets, and the like, or assaying for, ascertaining, establishing, or otherwise determining one or more factual characteristics of a viral disease, or similar conditions. The term encompasses diagnostic, prognostic, and monitoring applications for the ps20 polypeptides and/or ps20 polynucleotides and interferons or polynucleotides encoding interferons.

The invention also provides a method of assessing whether a patient is afflicted with or has a pre-disposition for a viral disease the method comprising comparing:
(a) levels of ps20 polypeptides or ps20 polynucleotides and one or more interferons or polynucleotides encoding interferons associated with the viral disease in a sample from the patient; and
(b) standard levels of ps20 polypeptides or ps20 polynucleotides and one or more interferons or polynucleotides encoding interferons in samples of the same type obtained from control samples, wherein altered levels of the ps20 polypeptides or the ps20 polynucleotides and one or more interferons or polynucleotides encoding interferons relative to the corresponding control levels of ps20 polypeptides or ps20 polynucleotides and one or more interferons or polynucleotides encoding interferons is an indication that the patient is afflicted with the viral disease.

In another particular embodiment of a method of the invention for assessing whether a patient is afflicted with or has a pre-disposition for a viral disease, higher levels of ps20 polypeptides or ps20 polynucleotides and lower levels of one or more interferons or polynucleotides encoding interferons in a sample relative to the corresponding standard levels is an indication that the patient is afflicted with or has a pre-disposition for a viral disease.

In an embodiment of the invention, a method for screening a subject for a viral disease is provided comprising (a) obtaining a biological sample from a subject; (b) detecting the amount of ps20 polypeptides and one or more interferons in said sample; and (c) comparing said amount of ps20 polypeptides and one or more interferons detected to a predetermined standard, where detection of levels of ps20 polypeptides and interferons that differ significantly from the standard indicates a viral disease. A significant difference between the levels of ps20 polypeptide levels and one or more interferon levels in a patient and the standard is an indication that the patient is afflicted with or has a predisposition to a viral disease. In an embodiment the amount of ps20 polypeptide(s) detected is greater than, and the amount of one or more interferon is less than, that of a standard and is indicative of a viral disease. In an embodiment, the levels of ps20 polypeptide or ps20 polynucleotide and one or more interferons are inversely correlated.

In another aspect, the invention provides a method for assessing the aggressiveness or indolence of a viral disease the method comprising comparing:
(a) levels of ps20 polypeptides or ps20 polynucleotides and one or more interferons or polynucleotides encoding interferons associated with the viral disease in a patient sample; and
(b) standard levels of the ps20 polypeptides or the ps20 polynucleotides and one or more interferons or polynucleotides encoding interferons in a control sample.

A significant difference between the levels in the sample and the standard levels is an indication that the viral disease is aggressive or indolent. In an embodiment, the levels of ps20 polypeptides are higher than standard levels and the levels of interferon are lower than standard levels. In an embodiment, the levels of ps20 polypeptide or ps20 polynucleotide and one or more interferons are inversely correlated.

In an aspect, the invention provides a method for monitoring the progression of a viral disease in a patient the method comprising:
(a) detecting ps20 polypeptides or ps20 polynucleotides and one or more interferons or polynucleotides encoding interferons associated with the disease in a sample from the patient at a first time point;
(b) repeating step (a) at a subsequent point in time; and
(c) comparing the levels detected in (a) and (b), and therefrom monitoring the progression of the viral disease.

The invention also provides a method for assessing the potential efficacy of a test agent for inhibiting a viral disease, and a method of selecting an agent for inhibiting a viral disease.

In an aspect, the invention contemplates a method of assessing the potential of a test compound to contribute to a viral disease comprising:
(a) maintaining separate aliquots of samples from a subject with high levels of ps20 polypeptides or ps20 polynucleotides and low levels of one or more interferons or polynucleotides encoding interferons compared to a control in the presence and absence of the test compound; and
(b) comparing the levels of ps20 polypeptides or ps20 polynucleotides and one or more interferons or polynucleotides encoding interferons associated with the disease in each of the aliquots.

A significant difference between the levels of ps20 polypeptides or ps20 polynucleotides and one or more interferons or polynucleotides encoding interferons in aliquots maintained in the presence of (or exposed to) the test compound relative to the aliquots maintained in the absence of the test compound, indicates that the test compound potentially contributes to a viral disease.

Certain methods of the invention employ binding agents (e.g. antibodies) that specifically recognize ps20 polypeptides and binding agents that specifically recognize interferons. In an aspect, the invention provides methods for determining the presence or absence or severity of a viral disease in a patient, comprising the steps of (a) contacting a biological sample obtained from a patient with one or more binding agent that specifically binds to one or more ps20 polypeptides and one or more binding agent that specifically binds to one or more interferons associated with the disease; and (b) detecting in the sample an amount of ps20 polypeptides that bind to a binding agent and interferons that bind to a binding agent, relative to predetermined standards or cut-off values, and therefrom determining the presence or absence or severity of a viral disease in the patient.

In another embodiment, the invention relates to a method for diagnosing and monitoring a viral disease in a subject by quantitating one or more ps20 polypeptides and one or more interferons associated with the disease in a biological sample from the subject comprising (a) reacting the biological sample with one or more binding agent specific for the ps20 polypeptides (e.g. an antibody) and one or more binding agent specific for the interferons that are directly or indirectly labelled with a detectable substance; and (b) detecting the detectable substance and thereby quantitating the ps20 polypeptides and interferons.

In another aspect the invention provides a method for using antibodies to detect expression of one or more ps20 polypeptides and one or more interferons in a sample, the method comprising: (a) combining antibodies specific for one or more ps20 polypeptides and antibodies specific for one or more interferons with a sample under conditions which allow formation of antibody:ps20 and antibody:interferon complexes; and (b) detecting complex formation, wherein complex formation indicates expression of the ps20 polypeptides and interferons in the sample. Expression may be compared with standards and is diagnostic of a viral disease.

In an embodiment quantitated levels of ps20 polypeptides and interferons are compared to levels quantitated for controls wherein an increase in ps20 polypeptide levels and a decrease in interferons compared with the controls are indicative of the viral disease.

Other methods of the invention employ one or more polynucleotides capable of hybridizing to one or more ps20 polynucleotides and polynucleotides encoding interferons. Thus, the present invention relates to a method for diagnosing and monitoring a viral disease in a sample from a subject comprising isolating nucleic acids, preferably mRNA, from the sample; and detecting ps20 polynucleotides and polynucleotides encoding interferons associated with the disease in the sample. The presence of different levels of ps20 polynucleotides and polynucleotides encoding interferons in the sample compared to a standard or control may be indicative of a positive or unfavorable prognosis.

In an embodiment of the invention, ps20 polynucleotide positive samples (e.g. higher levels of the ps20 polynucleotides compared to controls) and interferon polynucleotide negative samples (e.g. lower levels of one or more polynucleotides encoding interferons compared to controls) may be indicative of an unfavorable disease prognosis.

The invention provides methods for determining the presence or absence or severity of a viral disease in a subject comprising detecting in the sample levels of nucleic acids that hybridize to one or more ps20 polynucleotides and one or more polynucleotides encoding interferons, comparing the levels with a predetermined standard or cut-off value, and therefrom determining the presence or absence or severity of the viral disease in the subject. In an embodiment, the invention provides methods for determining the presence or absence or severity of a viral disease in a subject comprising (a) contacting a sample obtained from the subject with oligonucleotides that hybridize to one or more ps20 polynucleotides and oligonucleotides that hybridize to one or more polynucleotides encoding interferons; and (b) detecting in the sample levels of polynucleotides that hybridize to the oligonucleotides relative to a predetermined cut-off value, and therefrom determining the presence or absence or severity of the viral disease in the subject.

Within certain embodiments, the amount of polynucleotides that are mRNA are detected via polymerase chain reaction using, for example, oligonucleotide primers that hybridize to ps20 polynucleotides and primers that hybridize to polynucleotides encoding interferons, or complements of such polynucleotides. Within other embodiments, the amount of mRNA is detected using a hybridization technique, employing oligonucleotide probes that hybridize to ps20 polynucleotides and oligonucleotide probes that hybridize to polynucleotides encoding interferons, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of ps20 polynucleotides and polynucleotides encoding interferons in the sample. For mRNA the analyzing step may be accomplished using Northern Blot analysis to detect the presence of ps20 polynucleotides and polynucleotides encoding interferons. The analysis step may be further accomplished by quantitatively detecting the presence of ps20 polynucleotides and polynucleotides encoding interferons in the amplification product, and comparing the quantity of ps20 polynucleotides and polynucleotides encoding interferons detected against a panel of expected values for the known presence or absence of the ps20 polynucleotides and polynucleotides encoding interferons in control samples derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more ps20 polynucleotides and nucleic acid primers that hybridize to polynucleotides encoding interferons to produce amplification products; (d) analyzing the amplification products to detect an amount of mRNA encoding the ps20 polypeptides and polynucleotides encoding interferons; and (e) comparing the amount of mRNA to an amount detected against a panel of expected values for control and diseased samples derived using similar nucleic acid primers.

The invention also relates to kits for carrying out the methods of the invention. In an embodiment, the kit is for assessing whether a patient is afflicted with or has a pre-disposition to a viral disease and it comprises reagents for assessing one or more ps20 polypeptides or ps20 polynucleotides and one or more interferons or polynucleotides encoding interferons.

The invention contemplates the methods, compositions, and kits described herein using additional markers associated with a viral disease. The methods described herein may be modified by including reagents to detect the additional markers, or polynucleotides for the markers. In particular, the invention contemplates the methods described herein using multiple markers for a viral disease. Therefore, the invention contemplates a method for analyzing a biological sample for the presence of ps20 polypeptides or ps20 polynucleotides and one or more interferons or polynucleotides encoding the interferons, and other markers that are specific indicators of a viral disease. The methods described herein may be modified by including reagents to detect the additional markers, or nucleic acids for the additional markers.

The invention also provides a diagnostic composition comprising one or more ps20 polynucleotides and one or more polynucleotides encoding interferons. A composition is also provided comprising probes that specifically hybridize to ps20 polynucleotide(s) and probes that specifically hybridize to one or more polynucleotides encoding interferons, or fragments thereof. In an aspect, a composition is provided comprising one or more ps20 polynucleotide specific primer pairs and one or more interferon polynucleotide specific primer pairs capable of amplifying the polynucleotides using polymerase chain reaction methodologies. In an aspect, a composition is provided comprising antibodies specific for a ps20 polypeptide(s) and antibodies specific for interferons. The probes, primers or antibodies can be labeled with a detectable substance.

The invention relates to therapeutic applications for viral diseases. In an aspect, the invention provides a method for treating a viral disease or for reducing permissivity or susceptibility to a viral disease in a patient comprising administering to a patient in need thereof an effective amount of one or more interferons (e.g. an IFN-α), in particular, to modulate ps20 polypeptide levels in the patient. In an aspect of this method, one or more modulators, in particular antagonists, of a ps20 polypeptide or a ps20 polynucleotide are additionally administered to the patient. Antagonists of a ps20 polypeptide or a ps20 polynucleotide may be binding agents, in particular antibodies, specific for ps20 polypeptides.

The invention contemplates a method of using interferons in the preparation or manufacture of a medicament for modulating ps20 polypeptides or ps20 polynucleotides to reduce permissivity or susceptibility to a viral disease. In an aspect, the medicament additionally includes a modulator of a ps20 polypeptide or ps20 polynucleotide.

Another aspect of the invention is the use of ps20 polypeptides, peptides derived therefrom, or chemically produced (synthetic) peptides, or any combination of these molecules, for use in the preparation of vaccines to prevent a viral disease and/or to treat a viral disease. Therefore, the invention contemplates vaccines for stimulating or enhancing in a subject to whom the vaccine is administered production of antibodies directed against one or more ps20 polypeptides.

The invention provides an immunogenic composition for protecting subjects against a viral infection. An immunogenic composition of the invention comprises an immunogenic amount of a region of a ps20 polypeptide. In a composition of the invention, the region of a ps20 polypeptide defines an epitope which induces the formation of antibodies against ps20 thereby reducing viral permissivity. In embodiments of the invention an immunogenic composition comprises synthetic peptides about 5 to 200, 10 to 150, 10 to 100, 20 to 100, 10 to 50 or 20 to 25 amino acids in length which are portions of a ps20 polypeptide. In embodiments, the synthetic peptides are serotype specific peptides. Synthetic peptides may be used, for example, individually, in a mixture, or in a polypeptide or protein. For example, a polypeptide or protein can be created by fusing or linking the peptides to each other, synthesizing the polypeptide or protein based on the peptide sequences, and linking or fusing the peptides to a backbone. In addition, a liposome may be prepared with the peptides conjugated to it or integrated within it.

The invention also provides a method for stimulating or enhancing in a subject production of antibodies directed against one or more ps20 polypeptides. The method comprises administering to the subject an immunogenic composition or vaccine of the invention in a dose effective for stimulating or enhancing production of the antibodies.

The invention further provides a method for treating, preventing, or delaying recurrence of a viral disease. The method comprises administering to the subject a vaccine of the invention in a dose effective for treating, preventing, or delaying recurrence of a viral disease.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows graphs illustrating that exposure of cell populations that are highly susceptible to HIV, interferon alpha significantly reduces endogenous ps20 mRNA expression measured by qRT-PCR relative to a house-keeping gene.

FIG. 2 shows graphs illustrating that levels of ps20 gene expression distinguish between susceptible and resistant mice. Upper panel: the graph illustrates that at 48 hours post-infection, when A/J mice exhibit severe progressive pulmonary disease, gene expression levels for ps20 are significantly higher than expression levels expressed in the C57BL/6 mice. Lower panel: the graph illustrates that whereas ps20 gene expression levels in the infected and resistant C57BL/6 mice remained low throughout the course of disease, at 48 hours post-infection ps20 gene expression had increased >50-fold above basal expression in the hearts of infected and susceptible A/J mice.

FIG. 3 is a graph of M gene copy/ml versus hours post infection with H5N1 (A/Vt/3046/04) showing H5N1 infection in human lung tissue increases M gene copy number.

FIG. 4 is a graph showing relative fold induction of ps20 expression in Mock+PBS, Mock+IFN alfacon-1, H5N1+PBS and H5N1+IFN alfacon-1 treated human lung tissue. The data illustrate that H5N1 infection in human lung tissue increases ps20 gene expression, i.e., IFN treatment down-regulates ps20 expression in H5N1 infected human lung.

FIG. 5 is a graph showing relative fold induction of IFN-inducible antiviral ISGs (ISG15 and PKR1) gene expression in Mock+PBS, Mock+IFN alfacon-1, H5N1 (18 hours post-infection)+PBS and H5N1+IFN alfacon-1 treated human lung tissue.

FIG. 6 are graphs showing an enhanced IFN-α dependent antiviral response in cells lacking 4E-BPI or TSC2 from Kaur, S. et al, 2007 J. Biol. Chem, 1757-1768.

FIG. 7 is a graph showing 4E-BPI^{-/-} mice exhibit decreased susceptibility to infection with coxsackievirus B3 (CVB3).

FIG. 8 are graphs showing CVB3 infection induces enhanced expression of antiviral effectors (ISG15, PKR, and 2′5′OAS) in hearts of 4E-BPI^{-/-} mice.

FIG. 9 is a graph showing the relative expression of ps20 in wildtype versus 4E-BPI null mice.

### DETAILED DESCRIPTION OF THE INVENTION

Methods are provided for characterizing or detecting the presence of a viral disease in a sample, the absence of a viral disease in a sample, the stage of a viral disease, and other characteristics of viral diseases that are relevant to prevention, diagnosis, characterization, and therapy of viral diseases in a patient. Methods are also provided for assessing the efficacy of one or more test agents for inhibiting a viral disease, monitoring the progression of a viral disease, selecting an agent or therapy for inhibiting a viral disease, treating a patient afflicted with a viral disease, inhibiting a viral disease in a patient, and assessing the disease potential of a test agent.

### Glossary

In accordance with the present invention there may be employed conventional biochemistry, enzymology, molecular biology, microbiology, and recombinant DNA techniques within the skill of the art. Such techniques are explained fully in the literature. See for example, Sambrook et al, Molecular Cloning: A Laboratory Manual, Third Edition (2001) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. Thus, for example, reference to a composition containing “a modulator” includes a mixture of two or more modulators. The term “about” means plus or minus 0.1 to 50%, 5-50%, or 10-40%, preferably 10-20%, more preferably 10% or 15%, of the number to which reference is being made.

The terms “peptide”, “polypeptide” and “protein” are used interchangeably and as used herein refer to more than one amino acid joined by a peptide bond.

The term “effective amount” or “effective dose” refers to a non-toxic but sufficient amount of an agent (e.g. antibody or interferon) to provide the desired therapeutic or biological effect. The exact amount required will vary from subject to subject, depending on the species, age and general condition of the subject, the particular agent used, its mode of administration, and the like. An appropriate effective amount or effective dose may be determined by one or ordinary skill in the art using routine experimentation.

“Pharmaceutically acceptable” refers to a material that is not biologically or otherwise undesirable, i.e., the material may be administered to an individual without causing any undesirable biological effects or interacting in a deleterious manner with any of the other components of a composition in which it is contained.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art.

“Synthetic” refers to items, e.g., peptides or nucleic acids, which are not naturally occurring, in that they are isolated, synthesized or otherwise manipulated by man.

“Immunogenic” as used herein encompasses materials which are capable of producing an immune response.

“Composition” includes any composition of matter, including peptides, polypeptides, proteins, mixtures, vaccines, antibodies, or markers of the present invention.

“Detectable substances” include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), and predetermined polypeptide epitopes recognized by a secondary reporter (e.g., leucine zipper pair sequences, binding sites for secondary antibodies, metal binding domains, epitope tags).

“Viral diseases” means a class of diverse diseases and disorders caused by or believed to be caused by viruses. The term includes any stage of a viral infection, including incubation phase, latent or dormant phase, acute phase, and development and maintenance of immunity towards a virus. Consequently, the term “treatment’ is meant to include aspects of generating or restoring immunity of the patient's immune system, as well as aspects of suppressing or inhibiting virus activity. “Virus activity” includes virus replication, assembly, maturation, envelopment, extracellular virus formation, virus egress, and virus transmission. Viral diseases include, without limitation, human papilloma virus (HPV), HIV/AIDS, herpes, influenza, measles, polio, varicella-zoster, hepatitis A, hepatitis B, hepatitis C, hepatitis D, herpes simplex virus (type 1 and type 2), hepatitis E, hepatitis G, cytomegalovirus, meningitis, genital warts (HPV), a disease associated with respiratory syncytial virus infection, a disease associated with coxsackie virus infection, a disease associated with ebola virus infection, a disease associated with hantavirus infection, a disease associated with human papilloma virus infection, a disease associated with rotavirus infection, a disease associated with west nile virus infection, a disease associated with Epstein-Barr virus infection, a disease associated with papilloma virus infection, a disease associated with influenza virus infection, vesticular stomatitis virus infection, and dengue fever. The clinical sequelae of viral infections include without limitation, herpes, AIDS, lassa fever, kaposi's sarcoma, meningitis, mumps, polio, chicken pox, colds and flu, dengue fever, encephalitis, Fifth disease, shingles, genital warts, rubella, yellow fever, hepatitis A, B and C, measles, rabies, and smallpox. The singular form “viral disease” includes any one or more diseases selected from the class of viral diseases, and includes any compound or complex disease state wherein a component of the disease state includes a disease selected from the class of viral diseases.

In aspects of the invention the viral disease is a disease associated with a coronavirus, in particular a group II coronavirus. Examples of coronaviruses associated with viral diseases include without limitation, SARS coronavirus, coronavirus OC43, avian infectious bronchitis virus, Berne virus and hepatitis delta virus.

In other aspects of the invention, the viral disease is a disease associated with an enterovirus of the Picornaviridae family, in particular a coxsackievirus. Examples of viruses of the Picornaviridae family associated with disease include without limitation poliovirus 1, human rhinovirus 1A, hepatitis A virus, encephalomyocarditis virus, coxsackievirus group B virus and foot-and-mouth disease virus. In an embodiment, the viral disease is a disease associated with a coxsackievirus group B virus. In a particular embodiment, the viral disease is a CVB3 induced myocarditis.

In aspects of the invention, the viral disease is associated with or caused by an influenza virus. The term “influenza virus” refers to any strain of influenza virus that is capable of causing disease in an animal or human subject, or that is an interesting candidate for experimental analysis. [See Fields, B., et al., Fields' Virology, 4^{th}. ed., Philadelphia: Lippincott Williams and Wilkins; ISBN: 0781718325, 2001 for a description of influenza viruses.] Influenza viruses consist of three types, A, B, and C. Influenza A viruses infect a wide variety of birds and mammals, including humans, horses, pigs, ferrets, and chickens. Influenza B and C are present only in humans.

In aspects of the invention the viral disease is associated with or caused by any strain of influenza A virus that is capable of causing disease in an animal or human subject, or that is an interesting candidate for experimental analysis. A large number of influenza A isolates have been partially or completely sequenced (see Macken, C., Lu, H., Goodman, J., & Boykin, L., “The value of a database in surveillance and vaccine selection.” in Options for the Control of Influenza IV. A. D. M. E. Osterhaus, N. Cox & A. W. Hampson (Eds.) Amsterdam: Elsevier Science, 2001, 103-106; the Web site having URL http://www.flu.lanl.gov/).

In specific embodiments, the type A influenza virus is a subtype of the strain H1N1, H3N1, H5N1, H9N2, H7N2, H7N3 or H7N7.

In aspects of the invention, the viral disease is caused by an avian influenza virus, i.e. any influenza virus that may infect birds. In other aspects of the invention, the viral disease is associated with or caused by a highly pathogenic avian influenza virus (HPAI, i.e. an avian influenza virus that is highly virulent and characterized by high mortality). In one embodiment, the avian influenza virus is of the H5 subtype. In another embodiment, the avian influenza virus is of the H7 subtype. In another embodiment, the avian influenza virus is of the H5N1 subtype. In one embodiment, the avian influenza virus is A/Vietnam/1203/2004 (H5N1). In another embodiment, the avian influenza virus is A/Hong Kong/1 56/1996 (H5N1).

The terms “sample”, “biological sample”, and the like mean a material known or suspected of expressing or containing ps20 polypeptides or ps20 polynucleotides and interferons or polynucleotides encoding interferons. A test sample can be used directly as obtained from the source or following a pretreatment to modify the character of the sample. The sample can be derived from any biological source, such as tissues, extracts, or cell cultures, including cells (e.g. T cells, in particular CD4 T cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid, lavage fluid, and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like.

In an embodiment the sample is a human physiological fluid. In a particular embodiment, the sample is human serum. In a more particular embodiment, the sample comprises T cells, most particularly CD4 T cells. In another more particular embodiment, for example employing qRT-PCR, the sample comprises PBMCs, lung bronchial alveolar lavage or sputum.

The terms “subject” or “patient” refer to an animal including a warm-blooded animal such as a mammal, which is afflicted with or suspected of having or being pre-disposed to a viral disease. Mammal includes without limitation any members of the Mammalia. In general, the terms refer to a human. The terms also include animals bred for food, sport, or as pets, including domestic animals such as horses, cows, sheep, poultry, fish, pigs, and goats, and cats, dogs, and zoo animals, apes (e.g. gorilla or chimpanzee), and rodents such as rats and mice.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having substantial sequence identity. In an aspect a polypeptide variant has at least about 45%, preferably at least about 85%, more preferably at least about 90%, most preferably at least about 95% amino acid sequence identity with a native-sequence polypeptide. Polypeptide variants preferably retain the immunogenic activity of a corresponding native-sequence polypeptide. Particular polypeptide variants have at least 45%, preferably 50%, 55%, 60%, 65%, 70%, 75%, 80%, 85%, 90%, 95%, 97%, 98%, or 99% sequence identity to a native-sequence polypeptide. For example, a ps20 polypeptide variant may have at least 45%, preferably 50%, 55%, 60%, 65%, 70%, 75%, 80%, 85%, 90%, 95%, 97%, 98%, or 99% sequence identity to a sequence identified in Accession Nos. NP_{—}067020, EAW95486, EAW95487, AAG16647, AAG15263.1, Q9HC57, BAC11377.1, ABM84291.1, or ABM87681.1, or shown in SEQ ID NO. 2 and 3. Polypeptide variants also include, for instance, polypeptides wherein one or more amino acid residues are added to, or deleted from, the N- or C-terminus of the full-length or mature sequences of the polypeptide, including variants from other species, but excludes a native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

A variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion or fragment of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine polypeptide.

ps20 polypeptides also include chimeric or fusion proteins. A “chimeric protein” or “fusion protein” comprises all or part (preferably biologically active) of a ps20 polypeptide or interferon operably linked to a heterologous polypeptide (i.e., a polypeptide other than a ps20 polypeptide). Within the fusion protein, the term “operably linked” is intended to indicate that a ps20 polypeptide or interferon and the heterologous polypeptide are fused in-frame to each other. The heterologous polypeptide can be fused to the N-terminus or C-terminus of a ps20 polypeptide or an interferon. A useful fusion protein is a GST fusion protein in which a ps20 polypeptide or interferon is fused to the C-terminus of GST sequences. Another example of a fusion protein is an immunoglobulin fusion protein in which all or part of a ps20 polypeptide or interferon is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including but not limited to glycosylated, phosphorylated, acetylated, methylated or lapidated forms of the polypeptides. For example, an N-terminal methionine may be cleaved from a polypeptide, and a new N-terminal residue may or may not be acetylated.

The term “ps20 polypeptide” includes human ps20, in particular the native-sequence polypeptide, isoforms, chimeric polypeptides, all homologs, fragments, precursors, complexes, and modified forms and derivatives of human ps20. The amino acid sequence for native human ps20 includes the sequences of Accession Nos. NP_{—}067020, EAW95486, EAW95487, AAG16647, AAG15263.1, Q9HC57, BAC11377.1, ABM84291.1, or ABM87681.1 or shown in SEQ ID NO. 2 and 3.

ps20 polypeptides may be prepared by recombinant or synthetic methods, or isolated from a variety of sources, or by any combination of these and similar techniques.

“Interferon(s)” (also referred to as “IFN”) refers to a small, species-specific, single chain polypeptide, produced by cells in response to exposure to a variety of stimuli such as viruses, polypeptides, mitogens and the like. The term includes the human interferons including without limitation, leukocyte (interferon-alpha), and fibroblast (interferon-beta). [See The Interferon System (W. E. Stewart, II, Springer-Verlag, N.Y. 1979); Interferon Therapy (World Health Organization Technical Reports Series 676, World Health Organization, Geneva 1982, and DeMaeyer, E., et al., Interferons and Other Regulatory Cytokines, John Wiley and Sons, New York (1988 for general discussions of interferons]. The term includes native-sequence polypeptides, isoforms, chimeric polypeptides, homologs, fragments, precursors, and complexes. In aspects of the invention, the term relates to human interferon.

In aspects of the invention, the interferon compounds are alpha and beta interferons. “Alpha interferon” refers to a natural or recombinant interferon exhibiting biological properties similar to those of human leucocyte interferon. A number of alpha interferon species are known which are designated by a numeral after the Greek letter, and all are contemplated for use in this invention. Alpha hybrid interferons wherein fragments of two or more native alpha interferon species are joined (See for instance, EP No. 51873) may also be used in the present invention. Particular forms of alpha interferon for use in the present invention are IFN-α1, IFN-α2, IFN-α4, IFN-α5, IFNα6, IFNα7, IFNα8, IFN-αa10, IFN-α13, IFN-α14, IFN-α16, IFN-α17 and IFN-α21, preferably IFN-α1, IFN-α2, and IFN-α7. Alpha interferons may be prepared by recombinant-DNA methods [see, for example, Nagata et al., Nature, Vol. 284, pages 316-320 (1980)]. Beta interferon is a single species and may be prepared by recombinant-DNA methods.

In aspects of the invention, an interferon is a recombinant form. Recombinant DNA methods for producing recombinant interferons are known and are not intended to limit the invention in any way [See for example, U.S. Pat. Nos. 4,399,216, 5,149,636, 5,179,017 (Axel et al) and U.S. Pat. No. 4,470,461 (Kaufman)].

Examples of interferons that can be used in therapeutic applications of the invention include without limitation Roferon®, Intron®, Alferon®, Infergen®, Omniferon®, Alfacon-1, interferon-alpha, pegylated interferon-alpha, dimerized interferon, alpha, interferon-alpha conjugated to carriers, encapsulated interferon-alpha, interferon-alpha 12 as oral inhalant, interferon-alpha as injectable compositions, interferon-alpha as a topical composition, Roferon® analogues, Intron® analogues, Alferon® analogues, and Infergen® analogues, Omniferon® analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, encapsulated interferon-beta, interferon-beta as oral inhalant, interferon-beta as an injectable composition, interferon-beta as a topical composition, Avonex™ analogues, Betaseron™ analogues, Betaferon™ analogues, and Rebif™ analogues. The singular form, “interferon”, may mean any one or more compounds from the class of interferon compounds.

“ps20 polynucleotide(s)” refers to polynucleotides encoding ps20 polypeptides including native-sequence polypeptides, polypeptide variants including a portion of a polypeptide, an isoform, precursor, complex, a chimeric polypeptide, or modified forms and derivatives of the polypeptides. A polynucleotide encoding a native polypeptide employed in the present invention includes the polynucleotides encoding ps20 [e.g., Accession Nos. NM_{—}021197, AF169631, AAG16647.1, AF302109, AAG15263.1, AK075061, BAC11377.1, BC029159, AAH29159.1, AC010551.3, CH471114.2, AL713785, AL713785, CR595501, CR604862, CR608359, CR610530, CR615719, DQ893365.2, or DQ896682.2, Gene ID No. 58189, or SEQ ID NO. 1]. ps20 polynucleotides can be derived from sequences of polynucleotides encoding native ps20 polypeptides of various vertebrates, preferably mammals, and can be obtained using methods that are well-known to those having ordinary skill in the art

“Interferon polynucleotide(s)” or “polynucleotides encoding interferon(s)” refers to polynucleotides encoding interferons including native-sequence polypeptides, polypeptide variants including a portion of a polypeptide, an isoform, precursor, complex, a chimeric polypeptide, or modified forms and derivatives of the polypeptides. Interferon polynucleotides can be derived from wild-type interferon gene sequences of various vertebrates, preferably mammals and can be obtained using methods that are well-known to those having ordinary skill in the art [See, for example: U.S. Pat. No. 5,554,513 (DNA sequence encoding human interferon-beta2A); U.S. Pat. No. 5,541,312 (DNA encoding human fibroblast beta-2 interferon polypeptide); U.S. Pat. No. 5,231,176 (DNA encoding a human leukocyte interferon); U.S. Pat. No. 5,071,761 U.S. Pat. No. 4,970,161 (DNA encoding human interferon-gamma); U.S. Pat. No. 4,738,931 (DNA comprising a human interferon beta gene); U.S. Pat. No. 4,695,543 (human alpha-interferon Gx-1 gene) and U.S. Pat. No. 4,456,748 (DNA encoding sub-sequences of different, naturally, occurring leukocyte interferons).

ps20 polynucleotides and interferon polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

ps20 polynucleotides and interferon polynucleotides further include sequences that differ from a native sequence due to degeneracy in the genetic code. As one example, DNA sequence polymorphisms within the nucleotide sequence of a ps20 polypeptide or interferon may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

ps20 polynucleotides and interferon polynucleotides also include nucleic acids that hybridize under stringent conditions, preferably high stringency conditions to a ps20 polynucleotide or interferon polynucleotide, respectively. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

ps20 polynucleotides and interferon polynucleotides also include truncated nucleic acids or nucleic acid fragments and variant forms of the nucleic acids that arise by alternative splicing of an mRNA corresponding to a DNA.

The ps20 polynucleotides and interferon polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense ps20 polynucleotides).

A “significant difference” in levels of ps20 polypeptides, interferons, ps20 polynucleotides and/or interferon polynucleotides in a sample compared to a control or standard (e.g. predetermined levels in a standard or levels in other samples from a subject) may represent levels that are higher or lower than the standard error of the detection assay. In particular embodiments, the levels may be 1.5, 2, 3, 4, 5, or 6 times higher or lower than the control or standard.

The term “binding agent” refers to a substance that specifically binds to one or more ps20 polypeptides or interferons. A substance “specifically binds” to one or more selected polypeptides if it reacts at a detectable level with the polypeptides, and does not react detectably with peptides containing an unrelated or different sequence. Binding properties may be assessed using an ELISA, which may be readily performed by those skilled in the art (see for example, Newton et al., Develop. Dynamics 197: 1-13, 1993).

A binding agent may be a ribosome, with or without a peptide component, an aptamer, an RNA molecule, or a polypeptide. A binding agent may be a polypeptide that comprises one or more ps20 polypeptide sequence or interferon sequence, a peptide variant thereof, or a non-peptide mimetic of such a sequence. By way of example, a ps20 polypeptide sequence may be a peptide portion of a ps20 polypeptide that is capable of modulating a function mediated by a ps20 polypeptide.

An aptamer includes a DNA or RNA molecule that binds to nucleic acids and proteins. An aptamer that binds to a ps20 polypeptide or a ps20 polynucleotide or interferon or interferon polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to a ps20 polypeptide or an interferon.

An antibody also includes immunoglobulin types IgA, IgD, IgE, IgG, IgM and subtypes of any of the foregoing, wherein the light chains of the immunoglobulin may be kappa or lambda type.

Antibodies may be monospecific, bispecific, trispecific or of greater multispecificity. Multispecific antibodies may immunospecifically bind to different epitopes of a ps20 polypeptide or interferon, or may immunospecifically bind to both a ps20 polypeptide or interferon as well as a heterologous epitope, such as a heterologous polypeptide or solid support material.

In aspects of the invention, the antibodies immunospecifically bind to ps20 polypeptides or interferons including fragments thereof. Antibodies that immunospecifically bind to ps20 polypeptides or interferons include antibodies or fragments thereof that specifically bind to a ps20 polypeptide or interferon or fragments thereof and do not specifically bind to other non-ps20 polypeptides or non-interferons as the case may be. In embodiments of the invention, antibodies that immunospecifically bind to a ps20 polypeptide or interferon or fragments thereof do not non-specifically cross-react with other antigens (e.g., binding cannot be competed away with a non-ps20 polypeptide or non-interferon polypeptide as the case may be). Antibodies or fragments that immunospecifically bind to a ps20 polypeptide or interferons can be identified, for example, by immunoassays or other techniques known to those of skill in the art.

Antibodies may be from any animal origin including birds and mammals (e.g., human, murine, donkey, sheep, rabbit, goat, guinea pig, camel, horse, or chicken). In aspects of the invention, the antibodies are human or humanized monoclonal antibodies.

A “humanized antibody” includes forms of non-human antibodies that are chimeric antibodies which comprise minimal sequence derived from non-human immunoglobulin. Humanized antibodies may be human immunoglobulins (recipient antibody) in which hypervariable region residues of a recipient are replaced by hypervariable region residues from a non-human species (donor antibody) such as mouse, rat, rabbit or non-human primate having the desired specificity, affinity, and capacity. In some cases, Framework Region (FR) residues of the human immunoglobulin are replaced by corresponding non-human residues. In addition, humanized antibodies may comprise residues which are not found in the recipient antibody or in the donor antibody, for example, modifications to further refine antibody performance. A humanized antibody will typically comprise substantially all of at least one, and typically two, variable domains, in which all or substantially all of the hypervariable regions correspond to those of a non-human immunoglobulin and all or substantially all of the Framework Regions are those of a human immunoglobulin sequence. A humanized antibody may optionally comprise at least a portion of an immunoglobulin constant region (Fc), typically that of a human immunoglobulin that immunospecifically binds to a ps20 polypeptide that has been modified by the introduction of amino acid residue substitutions, deletions or additions (i.e., mutations). In aspects of the invention, a humanized antibody is a derivative that comprises amino acid residue substitutions, deletions or additions in one or more non-human CDRs. A derivative may have substantially the same binding, better binding, or poorer binding when compared to a non-derivative humanized antibody. [See the following for details of humanized antibodies: U.S. Pat. Nos. 5,225,539, 5,530,101, 5,565,332, 5,585,089, 5,766,886, and 6,407,213; and Padlan, 1991, Molecular Immunology 28(4/5):489-498; Studnicka et al., 1994, Protein Engineering 7(6):805-814; Roguska et al., 1994, PNAS 91:969-973; Tan et al., 2002, J. Immunol. 169:1119-25; Caldas et al., 2000, Protein Eng. 13:353-60; Morea et al., 2000, Methods 20:267-79; Baca et al., 1997, J. Biol. Chem. 272:10678-84; Roguska et al., 1996, Protein Eng. 9:895-904; Couto et al., 1995, Cancer Res. 55 (23 Supp):5973s.sup.-5977s; Couto et al., 1995, Cancer Res. 55:1717-22; Sandhu, 1994, Gene 150:409-10; Pedersen et al., 1994, J. Mol. Biol. 235:959-73; Jones et al., 1986, Nature 321:522-525; Reichmann et al., 1988, Nature 332:323-329; and Presta, 1992, Curr. Op. Struct. Biol. 2:593-596.

Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for ps20 polypeptides or interferons may also be obtained from scientific or commercial sources. In an embodiment of the invention, antibodies are reactive against ps20 polypeptides or interferons if they bind with a Kₐ of greater than or equal to 10⁻⁷ M.

In aspects of the invention, the antibody is a purified antibody. By “purified” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

“Microarray” and “array,” refer to nucleic acid or nucleotide arrays or protein or peptide arrays that can be used to detect biomolecules associated with a viral disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-bases long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known (see for example, Zhu et al., Science 293:2101 (2001).

“Synergistic” means a greater pharmacological or therapeutic effect with the use of a multi-component composition or combination therapy of an interferon and a modulator of a ps20 polypeptide or ps20 polynucleotide, such as an antagonist of a ps20 polypeptide or ps20 polynucleotide, than with the use of any of these treatments alone. This synergistic effect can work through either similar or different mechanisms or pathways of action. One potential advantage of a combination therapy with a synergistic effect is that standard dosages may be used for a greater therapeutic effect than expected; or alternatively lower dosages or reduced frequency of administration of the treatments may be used to achieve a better therapeutic effect.

A “combination treatment” and “administering in combination” mean that the active ingredients are administered concurrently to a patient being treated. When administered in combination each component may be administered at the same time, or sequentially in any order at different points in time. Therefore, each component may be administered separately, but sufficiently close in time to provide the desired effect, in particular a synergistic effect. The first compound may be administered in a regimen that additionally comprises treatment with the second compound. In aspects the terms refer to the administration of an interferon and a modulator of a ps20 polypeptide or ps20 polynucleotide, such as an antagonist of a ps20 polypeptide or ps20 polynucleotide, including separate administration of medicaments each containing one of the compounds as well as simultaneous administration whether or not the compounds are combined in one formulation or whether they are in separate formulations.

### Nucleic Acid Methods/Assays

A viral disease may be detected based on the amount/level of ps20 polynucleotides and one or more polynucleotides encoding interferons in a sample. Techniques for detecting polynucleotides such as polymerase chain reaction (PCR) and hybridization assays are well known in the art.

Probes may be used in hybridization techniques to detect ps20 polynucleotides and one or more polynucleotides encoding interferons. The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of ps20 polynucleotides, preferably they comprise 10-150, more particularly 10-30, 10-40, 20-50, 40-80, 50-150, 80-120 nucleotides in length. The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect ps20 polynucleotides or interferon polynucleotides. The nucleotide probes may also be useful in the diagnosis of a viral disease involving one or more ps20 polynucleotides, in monitoring the progression of such disorder, or monitoring a therapeutic treatment.

The detection of ps20 polynucleotides and polynucleotides encoding interferons may involve the amplification of specific gene sequences using an amplification method such as polymerase chain reaction (PCR), followed by the analysis of the amplified molecules using techniques known to those skilled in the art. Standard procedures for polymerase chain reaction (PCR) amplification of genomic DNA or cloned sequences may be utilized. (See, for example, in Innis et al., eds., 1990, PCR Protocols: A Guide to Methods and Applications, Academic Press Inc., San Diego, Calif.). Controlled robotic systems may be useful for isolating and amplifying nucleic acids.

Suitable primers can be routinely designed by one of skill in the art. Computer programs known in the art can be used to design primers with the required specificity and optimal amplification properties, such as Oligo version 5.0 (National Biosciences). By way of example, at least two oligonucleotide primers may be employed in a PCR based assay to amplify a portion of a polynucleotide encoding one or more ps20 polynucleotides derived from a sample, wherein at least one of the oligonucleotide primers is specific for (i.e. hybridizes to) a polynucleotide encoding a ps20 polypeptide. Amplified cDNA can be separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a ps20 polynucleotide or interferon polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of ps20 polynucleotide or interferon polynucleotide expression. For example, RNA may be isolated from a cell type known to express a ps20 polynucleotide and/or polynucleotide encoding an interferon and tested utilizing the hybridization (e.g. standard Northern analyses) or PCR techniques referred to herein.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a ps20 polynucleotide or interferon polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by for example, ethidium bromide staining and agarose gel electrophoresis.

Amplification may be performed on samples obtained from a subject with a suspected viral disease and an individual who is not afflicted with a viral disease. The reaction may be performed on several dilutions of cDNA spanning at least two orders of magnitude. A statistically significant difference in expression in several dilutions of the subject sample as compared to the same dilutions of the non-disease sample may be considered positive for the presence of a viral disease.

In an embodiment, the invention provides methods for determining the presence or absence of a viral disease in a subject comprising (a) contacting a sample obtained from the subject with oligonucleotides that hybridize to ps20 polynucleotides and oligonucleotides that hybridize to one or more polynucleotides encoding interferons; and (b) detecting in the sample a level of nucleic acids that hybridize to the polynucleotides relative to a predetermined cut-off value, and therefrom determining the presence or absence of a viral disease in the subject.

The invention provides a method wherein a ps20 polynucleotide or interferon polynucleotide mRNA is detected by (a) isolating mRNA from a sample and combining the mRNA with reagents to convert it to cDNA; (b) treating the converted cDNA with amplification reaction reagents and nucleic acid primers that hybridize to one or more ps20 polynucleotides or interferon polynucleotides, to produce amplification products; (d) analyzing the amplification products to detect amounts of mRNA encoding ps20 polypeptides or interferons; and (e) comparing the amount of mRNA to an amount detected against a panel of controls derived using similar nucleic acid primers. Higher levels of ps20 polynucleotides and lower levels of interferon polynucleotides in patients compared to controls can be indicative of an unfavorable disease prognosis.

In another embodiment, the invention provides methods for determining the presence or absence of a viral disease in a subject comprising (a) contacting a sample obtained from the subject with oligonucleotides that hybridize to one or more ps20 polynucleotides and one or more interferon polynucleotides; and (b) detecting in the sample levels of nucleic acids that hybridize to the polynucleotides relative to a predetermined cut-off value, and therefrom determining the presence or absence of a viral disease in the subject. In particular, the invention provides a method wherein WFDC1 and an IFN-alpha mRNA is detected by (a) isolating mRNA from a sample and combining the mRNA with reagents to convert it to cDNA; (b) treating the converted cDNA with amplification reaction reagents and nucleic acid primers that hybridize to WFDC1 or an IFN-alpha polynucleotide to produce amplification products; (d) analyzing the amplification products to detect an amount of WFDC1 or IFN-alpha mRNA; and (e) comparing the amount of mRNA to an amount detected against a panel of expected values for healthy individuals derived using similar nucleic acid primers. Patient samples that comprise higher levels, in particular significantly higher levels, of WFDC1 and lower levels of IFN-alpha polynucleotides compared to a control sample are indicative of a viral disease.

Oligonucleotides or longer fragments derived from ps20 polynucleotides and interferon polynucleotides may be used as targets in a microarray. The microarray can be used to simultaneously monitor the expression levels of large numbers of genes and to identify genetic variants and mutations. The information from the microarray may be used to determine gene function, to diagnose a viral disease, and to develop and monitor the activities of therapeutic agents. The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus, the invention also includes an array comprising one or more ps20 polynucleotides, one or more polynucleotides encoding interferons and optionally other markers. The array can be used to assay expression of ps20 polynucleotides and one or more polynucleotides encoding interferons in the array. The invention allows the quantitation of expression of one or more ps20 polynucleotides and one or more polynucleotides encoding interferons.

Microarrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the ps20 polynucleotides and one or more polynucleotides encoding interferons. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, about 20 and about 100 bases, or about 40 and about 70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are produced by synthesizing polynucleotide probes on a support wherein the nucleotide probes are attached to the support covalently at either the 3′ or the 5′ end of the polynucleotide.

The invention provides microarrays comprising a disclosed marker set. In one embodiment, the invention provides a microarray for distinguishing viral disease samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the different nucleotide sequences comprising ps20 polynucleotides and one or more polynucleotides encoding interferons.

The invention provides gene marker sets that distinguish viral disease and uses thereof. In an aspect, the invention provides a method for classifying a viral disease comprising detecting a difference in the expression of a first plurality of genes relative to a control, the first plurality of genes comprising ps20 polynucleotides and polynucleotides encoding interferons. In another specific aspect, the control comprises nucleic acids derived from a pool of samples from individual control patients.

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a viral disease in a subject may be determined by (a) contacting a sample from the subject with a binding agent; (b) detecting in the sample a level of a ps20 polypeptide and one or more interferons that binds to the binding agent; and (c) comparing the level of protein with a predetermined standard or cut-off value.

In particular embodiments of the invention, the binding agent is an antibody. Antibodies specifically reactive with one or more ps20 polypeptide, or one or more interferons or derivatives, such as enzyme conjugates or labeled derivatives, may be used to detect one or more ps20 polypeptide and one or more interferons in various samples (e.g. biological materials). They may be used as diagnostic or prognostic reagents and they may be used to detect abnormalities in the levels of one or more ps20 polypeptide and one or more interferons, and/or temporal, tissue, cellular, or subcellular location of one or more ps20 polypeptides and one or more interferons. Antibodies may also be used to screen potentially therapeutic compounds in vitro to determine their effects on viral diseases involving one or more ps20 polypeptides and other conditions. In vitro immunoassays may also be used to assess or monitor particular therapies.

In an aspect, the invention provides a method for monitoring or diagnosing a viral disease in a subject by quantitating one or more ps20 polypeptides and one or more interferons in a biological sample from the subject comprising reacting the sample with antibodies specific for one or more ps20 polypeptides and antibodies specific for one or more interferons, which are directly or indirectly labeled with detectable substances and detecting the detectable substances. In a particular embodiment of the invention, ps20 polypeptides and one or more interferons are quantitated or measured.

In an aspect of the invention, a method for detecting a viral disease is provided comprising:
(a) providing a sample suspected of containing one or more ps20 polypeptides and one or more interferons associated with a viral disease;
(b) contacting said sample with antibodies that specifically bind to the ps20 polypeptides and antibodies that specifically bind to one or more interferons under conditions effective to bind the antibodies and form complexes;
(c) measuring the amount of ps20 polypeptides and interferons present in the sample by quantitating the amount of the complexes; and
(d) comparing the amount of ps20 polypeptides and interferons present in the samples with the amount of ps20 polypeptides in a control, wherein a change or significant difference in the amount of ps20 polypeptides and interferons in the sample compared with the amount in the control is indicative of a viral disease.

In an embodiment, the invention contemplates a method for monitoring the progression of a viral disease in an individual, comprising:
(a) contacting antibodies which bind to one or more ps20 polypeptides and antibodies that bind to one or more interferons with a sample from the individual so as to form complexes comprising the antibodies and ps20 polypeptides and antibodies and interferons in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the disease in said individual.

The amount of complexes may also be compared to a value representative of the amount of the complexes from an individual not afflicted with a viral disease at different stages. A significant difference in complex formation may be indicative of an unfavourable prognosis.

In embodiments of methods of the invention, a ps20 polypeptide of SEQ ID NO. 2 or 3 is detected in samples and higher levels, in particular significantly higher levels, compared to a control is indicative of a viral disease. In embodiments of the invention, IFN-α2, IFN-α1 and IFNα7 is detected in samples and lower levels, in particularly significantly lower levels compared to a control is indicative of a viral disease.

Antibodies may be used in any known immunoassays that rely on the binding interaction between antigenic determinants of ps20 polypeptide and interferons and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of ps20 polypeptides and interferons in a sample may be accomplished by competitive or noncompetitive immunoassay procedures in either a direct or indirect format. Detection of ps20 polypeptides and interferons using antibodies can be done utilizing immunoassays which are run in either the forward, reverse or simultaneous modes. An immunoassay method may be competitive or noncompetitive. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to an embodiment of the invention, an immunoassay for detecting one or more ps20 polypeptides and one or more interferons in a biological sample comprises contacting binding agents that specifically bind to ps20 polypeptides and binding agents that specifically bind to one or more interferons in the sample under conditions that allow the formation of first complexes comprising a binding agent and ps20 polypeptides and one or more interferons and determining the presence or amount of the complexes as a measure of the amount of ps20 polypeptides and one or more interferons contained in the sample. In a particular embodiment, the binding agents are labeled differently or are capable of binding to different labels.

Antibodies may be used to detect and quantify one or more ps20 polypeptides and one or more interferons in a sample in order to diagnose a viral disease. Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a sample obtained from a subject suspected of having a viral disease is contacted with antibodies, preferably monoclonal antibodies recognizing one or more ps20 polypeptides and monoclonal antibodies recognizing one or more interferons. The binding of antibodies to ps20 polypeptides and interferons is determined by selective staining of the sample by standard immunohistochemical procedures. The same procedure may be repeated on the same sample using other antibodies that recognize ps20 polypeptides and/or interferons. Alternatively, a sample may be contacted with antibodies against one or more ps20 polypeptides and antibodies against one or more interferons simultaneously, provided that the antibodies are labeled differently or are able to bind to a different label.

An antibody microarray in which binding sites comprise immobilized, preferably monoclonal, antibodies specific to a substantial fraction of ps20 polypeptides and interferons of interest can be utilized in the present invention. Antibody arrays can be prepared using methods known in the art [(see for example, Zhu et al., Science 293:2101 (2001) and reference 20].

Binding agents (e.g., antibodies) specific for one or more ps20 polypeptides or one or more interferons may be labelled with a detectable substance and detected in samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups, and predetermined polypeptide epitopes recognized by a secondary reporter. In some embodiments, labels are attached via spacer arms of various lengths to reduce potential steric hindrance. Antibodies may also be coupled to electron dense substances, such as ferritin or colloidal gold, which are readily visualized by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steroid isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for antibodies reactive against ps20 polypeptides or interferons. By way of example, if the antibody having specificity against one or more ps20 polypeptide is a rabbit IgG antibody, the second antibody may be goat anti-rabbit gamma-globulin labelled with a detectable substance as described herein.

ps20 polypeptide antibodies or interferon antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for a ps20 polypeptide antibody is labeled with an enzyme.

Methods for conjugating or labelling the binding agents (e.g. antibodies) discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,”Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more ps20 polypeptides and one or more interferons. Generally, antibodies may be labeled with detectable substances and one or more ps20 polypeptides or one or more interferons may be localized in tissues and cells based upon the presence of the detectable substances.

In the context of the methods of the invention, the sample, binding agents (e.g. antibodies against ps20 polypeptides or antibodies against interferons), or ps20 polypeptides or interferons may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized binding agent (e.g. antibody) may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a ps20 polypeptide may be immobilized using sheep anti-mouse IgG Fc fragment specific antibody coated on the carrier or support.

Where a radioactive label is used as a detectable substance, ps20 polypeptides or interferons may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

### Computer Systems

Analytic methods contemplated herein can be implemented by use of computer systems and methods described below and known in the art. Thus, the invention provides computer readable media comprising one or more ps20 polypeptides, ps20 polynucleotides and one or more interferons or polynucleotides encoding interferons, and optionally other markers and information related thereto. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls including ps20 polypeptides, ps20 polynucleotides, interferons, and interferon polynucleotides.

“Recorded” refers to a process for storing information on computer readable medium. The skilled artisan can readily adopt any of the presently known methods for recording information on computer readable medium to generate manufactures comprising information on one or more ps20 polypeptides, ps20 polynucleotides and one or more interferons or polynucleotides encoding interferons, and optionally other markers.

A variety of data processor programs and formats can be used to store information on one or more ps20 polypeptides, ps20 polynucleotides and one or more interferons or polynucleotides encoding interferons and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a viral disease, comprising determining the presence or absence of one or more ps20 polypeptides, ps20 polynucleotides and one or more interferons or polynucleotides encoding interferons, and optionally other markers, and based on the presence or absence of the ps20 polypeptides, ps20 polynucleotides and interferons or polynucleotides encoding interferons and optionally other markers, determining a viral disease, and optionally recommending a procedure or treatment.

In an aspect of the invention a method is provided for detecting a viral disease using a computer having a processor, memory, display, and input/output devices, the method comprising the steps of:
(a) creating records of one or more ps20 polypeptides or ps20 polynucleotides and one or more interferons or polynucleotides encoding interferons, and optionally other markers of the viral disease in a sample suspected of containing one or more ps20 polypeptides, ps20 polynucleotides and one or more interferons or polynucleotides encoding interferons or optionally other markers;
(b) providing a database comprising records of data comprising one or more ps20 polypeptides, ps20 polynucleotides and one or more interferons or polynucleotides encoding interferons, and optionally other markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a sample of an individual with a viral disease.

In an aspect of the invention, the computer systems, components, and methods described herein are used to monitor a viral disease or determine the stage of a viral disease.

### Methods for Identifying or Evaluating Substances/Compounds

The invention contemplates methods designed to identify substances that modulate the biological activity of a ps20 polypeptide including substances that bind to a ps20 polypeptide or portion thereof, or bind to other proteins that interact with a ps20 polypeptide, to compounds that interfere with, or enhance the interaction of a ps20 polypeptide and substances that bind to a ps20 polypeptide, or other proteins that interact with a ps20 polypeptide. Methods can also be utilized that identify compounds that bind to regulatory sequences of a ps20 polynucleotide.

Substances, agents and compounds that may be identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], polynucleotides (e.g., siRNA) and small organic or inorganic molecules. The substance, agent or compound may be an endogenous physiological compound or it may be a natural or synthetic compound.

Substances identified using the methods of the invention may be isolated, cloned and sequenced using conventional techniques. A substance that associates with a ps20 polypeptide of the invention may be an agonist or antagonist of the biological or immunological activity of the polypeptide. The term “agonist”, refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which modulate a ps20 polypeptide can be identified based on their ability to bind to a ps20 polypeptide. Therefore, the invention also provides methods for identifying substances which bind to a ps20 polypeptide. Substances which can bind with a ps20 polypeptide may be identified by reacting a ps20 polypeptide with a test substance which potentially binds to a ps20 polypeptide, under conditions which permit the formation of substance-ps20 polypeptide complexes and removing and/or detecting the complexes. The complexes can be detected by assaying for substance-ps20 polypeptide complexes, for free substance, or for non-complexed ps20 polypeptide. Conditions which permit the formation of substance-ps20 polypeptide complexes may be selected having regard to factors such as the nature and amounts of the substance and the polypeptide. The substance-protein complex, free substance or non-complexed polypeptides may be isolated by conventional isolation techniques, for example, salting out, chromatography, electrophoresis, gel filtration, fractionation, absorption, polyacrylamide gel electrophoresis, agglutination, or combinations thereof To facilitate the assay of the components, antibody against ps20 polypeptide or the substance, or labelled ps20 polypeptide, or a labelled substance may be utilized. The antibodies, polypeptides, or substances may be labelled with a detectable substance as described above.

A ps20 polypeptide, or the substance used in these methods of the invention may be insolubilized. For example, a ps20 polypeptide, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized polypeptide or substance may be prepared by reacting the material with a suitable insoluble carrier using known chemical or physical methods, for example, cyanogen bromide coupling.

The invention also contemplates a method for evaluating a compound for its ability to modulate the biological activity of a ps20 polypeptide by assaying for an agonist or antagonist (i.e., enhancer or inhibitor) of the binding of a ps20 polypeptide with a substance which binds with a ps20 polypeptide. The basic method for evaluating if a compound is an agonist or antagonist of the binding of a ps20 polypeptide and a substance that binds to the polypeptide is to prepare a reaction mixture containing the ps20 polypeptide and the substance under conditions which permit the formation of substance—ps20 polypeptide complexes, in the presence of a test compound. The test compound may be initially added to the mixture, or may be added subsequent to the addition of the ps20 polypeptide and substance. Control reaction mixtures without the test compound or with a placebo are also prepared. The formation of complexes is detected, and the formation of complexes in the control reaction but not in the reaction mixture indicates that the test compound interferes with the interaction of the ps20 polypeptide and substance. The reactions may be carried out in the liquid phase or the ps20 polypeptide, substance, or test compound may be immobilized as described herein. The ability of a compound to modulate the biological activity of a ps20 polypeptide may be tested by determining the biological effects on cells.

It will be understood that the agonists and antagonists, i.e., inhibitors and enhancers, that can be assayed using the methods of the invention may act on one or more of the binding sites on the polypeptide or substance including agonist binding sites, competitive antagonist binding sites, non-competitive antagonist binding sites or allosteric sites.

The invention also makes it possible to screen for antagonists that inhibit the effects of an agonist of the interaction of a ps20 polypeptide with a substance which is capable of binding to the ps20 polypeptide. Thus, the invention may be used to assay for a compound that competes for the same binding site of a ps20 polypeptide.

The invention also contemplates methods for identifying compounds that bind to a ps20 polypeptide or proteins that interact with a ps20 polypeptide. Protein-protein interactions may be identified using conventional methods such as co-immunoprecipitation, crosslinking and co-purification through gradients or chromatographic columns. Methods may also be employed that result in the simultaneous identification of genes which encode proteins interacting with a ps20 polypeptide. These methods include probing expression libraries with labeled ps20 polypeptide.

Two-hybrid systems may also be used to detect protein interactions in vivo. Generally, plasmids are constructed that encode two hybrid proteins. A first hybrid protein consists of the DNA-binding domain of a transcription activator protein fused to a ps20 polypeptide, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the methods described herein. In particular, ps20 polypeptides fused to a glutathione-S-transferase may be used in the methods.

A modulator of a ps20 polypeptide of the invention may also be identified based on its ability to inhibit or enhance activity of the polypeptide. In aspects of the invention, substances that modulate ps20 polypeptides can be selected by assaying for a substance that inhibits or stimulates, preferably inhibits, the activity of a ps20 polypeptide. Such a substance can be identified based on its ability to specifically interfere with or stimulate, preferably interfere with, the activity of a ps20 polypeptide.

The invention also contemplates methods for evaluating test agents or compounds for their ability to reduce or inhibit viral infection or disease. Therefore, the invention provides a method for assessing the potential efficacy of a test agent for reducing or inhibiting a viral infection in a patient, the method comprising comparing:
(a) levels of one or more ps20 polypeptides, ps20 polynucleotides, interferons and interferon polynucleotides in a sample obtained from a patient and exposed to the test agent; and
(b) levels of one or more ps20 polypeptides, ps20 polynucleotides, interferons and interferon polynucleotides in a second sample obtained from the patient, wherein the sample is not exposed to the test agent, wherein a significant difference in the levels of expression of one or more ps20 polypeptides, ps20 polynucleotides, interferons and interferon polynucleotides relative to the second sample, is an indication that the test agent is potentially efficacious for reducing or inhibiting a viral infection in the patient.

The first and second samples may be portions of a single sample obtained from a patient or portions of pooled samples obtained from a patient.

In an aspect, the invention provides a method of selecting an agent for inhibiting a viral disease in a patient comprising:
(a) providing a sample from the patient;
(b) separately maintaining aliquots of the sample in the presence of a plurality of test agents;
(c) comparing one or more ps20 polypeptides, ps20 polynucleotides, interferons and/or interferon polynucleotides in each of the aliquots; and
(d) selecting one of the test agents which alters the levels of one or more ps20 polypeptides, ps20 polynucleotides, interferons and/or interferon polynucleotides relative to other test agents.

Still another aspect of the present invention provides a method of conducting a drug discovery business comprising:
(a) providing one or more methods or assay systems for identifying agents that reduce or inhibit a viral infection in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to a viral disease (e.g. HIV or influenza) comprising:
(a) maintaining separate aliquots of cells (e.g. CD4 T cells) from a patient with a viral disease in the presence and absence of the test compound; and
(b) comparing one or more ps20 polypeptides, ps20 polynucleotides, interferons and/or interferon polynucleotides in each of the aliquots.

A significant difference between the levels of the ps20 polypeptides, ps20 polynucleotides, interferons and/or interferon polynucleotides in the aliquot maintained in the presence of (or exposed to) the test compound relative to the aliquot maintained in the absence of the test compound, indicates that the test compound possesses the potential to contribute to a viral disease.

### Kits

The invention also contemplates kits for carrying out the methods of the invention. Kits may typically comprise components required for performing a diagnostic assay. Components include but are not limited to compounds, reagents, containers, and/or equipment.

The invention contemplates a kit for assessing the presence of ps20 polypeptides and interferons, wherein the kit comprises antibodies specific for one or more ps20 polypeptides and antibodies specific for one or more interferons, or primers or probes for polynucleotides encoding same, and optionally probes, primers or antibodies specific for other markers associated with a viral disease.

The methods described herein may be performed by utilizing pre-packaged diagnostic kits comprising one or more ps20 polynucleotide and interferon polynucleotide or antibodies specific for ps20 polypeptides or interferons, which may be conveniently used, e.g., in clinical settings to screen and diagnose patients and to screen and identify those individuals exhibiting a predisposition to developing a viral disease.

In an embodiment, a container with a kit comprises a binding agent as described herein. By way of example, the kit may contain antibodies or antibody fragments which bind specifically to epitopes of one or more ps20 polypeptides and optionally other markers (e.g. interferons), antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more ps20 polypeptide, antibodies or fragments of antibodies which bind specifically to an epitope of one or more interferons and means for detecting binding of the antibodies to their epitopes, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use usually do not require other reagents, as for example, where the kit contains a radiolabelled antibody preparation for in vivo imaging.

A kit may be designed to detect the levels of ps20 polynucleotides and interferon polynucleotides in a sample. Such kits generally comprise oligonucleotide probes or primers, as described herein, that hybridize to ps20 polynucleotides and polynucleotides encoding one or more interferon. Such oligonucleotides may be used, for example, within a PCR or hybridization procedure. Additional components that may be present within the kits include second oligonucleotides and/or diagnostic reagents or a container to facilitate detection of the polynucleotides.

The invention provides a kit containing a microarray described herein ready for hybridization to target ps20 polynucleotides and interferon polynucleotides, plus software for the analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting a viral disease in a patient. The kit comprises reagents for assessing one or more ps20 polypeptides, one or more interferons, and optionally a plurality of test agents or compounds.

### Therapeutic Applications

ps20 polypeptide specific antibodies and polynucleotides specific for ps20 (e.g., siRNA), disclosed herein and modulators identified by the methods described herein may be used in the treatment of viral diseases. The antibodies and modulators may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to living organisms including humans and animals. Administration of a therapeutically effective amount of an active ingredient or pharmaceutical composition disclosed herein is defined as an amount effective, at dosages and for periods of time necessary to achieve the desired result. For example, a therapeutically effective amount of a substance may vary according to factors such as the disease state, age, and weight of the individual, and the ability to elicit a desired response in the individual. Dosage regima may be adjusted to provide the optimum therapeutic response. For example, several divided doses may be administered daily or the dose may be proportionally reduced as indicated by the exigencies of the therapeutic situation.

An active therapeutic substance described herein may be administered in a convenient manner including without limitation by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents (e.g. antivirals) or other forms of treatment. The compositions of the invention may be administered concurrently, separately, or sequentially with other therapeutic agents or therapies (e.g. interferons).

A composition of the invention may contain at least one modulator of a ps20 polypeptide or ps20 polynucleotide, in particular, at least one antagonist of a ps20 polypeptide or a ps20 polynucleotide, or substance or modulator identified in accordance with the methods of the invention, alone or together with other therapeutic agents or active substances. The compositions of the invention may be administered together with or prior to administration of other agents such as biological factors that have been found to affect reduce or inhibit viral diseases. Examples of biological factors include the interferons, in particular Type I interferons, more particularly an IFN-alpha.

In aspects of the invention, compositions, conjugates, and methods (e.g. combination therapies) are provided comprising one or more modulator of ps20 polypeptides or polynucleotides, in particular one or more antagonists of a ps20 polypeptide or a ps20 polynucleotide, including in particular ps20 polypeptide specific antibodies, polynucleotides specific for ps20 (e.g., siRNA) and substances or modulators identified by the methods described herein, and one or more interferon. In an aspect these compositions, conjugates, and methods provide therapeutic effects in the treatment of viral diseases for which interferons have a therapeutic effect. The invention relates to compositions, conjugates, and methods for the prevention, intervention, and/or treatment of a viral disease comprising a therapeutically effective amount of an interferon and a modulator of ps20 polypeptides or polynucleotides, in particular a therapeutically effective amount that provides desirable therapeutic effects. The invention also relates to a use of one or more antagonists of a ps20 polypeptide or a ps20 polynucleotide, including in particular ps20 polypeptide specific antibodies, polynucleotides specific for ps20 (e.g., siRNA) and substances or modulators identified by the methods described herein, and one or more interferon for the prevention, intervention and/or treatment of a viral disease. The invention also includes one or more antagonists of a ps20 polypeptide or a ps20 polynucleotide, including in particular ps20 polypeptide specific antibodies, polynucleotides specific for ps20 (e.g., siRNA) and substances or modulators identified by the methods described herein, and one or more interferon in the manufacture of a medicament. The invention also includes one or more antagonists of a ps20 polypeptide or a ps20 polynucleotide, including in particular ps20 polypeptide specific antibodies, polynucleotides specific for ps20 (e.g., siRNA) and substances or modulators identified by the methods described herein, and one or more interferon for the prevention, intervention and/or treatment of a viral disease.

A composition, conjugate, or method (e.g. combination therapy) comprising an interferon and a modulator of ps20 polypeptides or polynucleotides employing different mechanisms to achieve maximum therapeutic efficacy, may improve tolerance to the therapy with a reduced risk of side effects that may result from higher doses or longer term monotherapies (i.e. therapies with each compound alone). A treatment of the invention can permit the use of lower doses of each compound with reduced adverse effects of each compound. A suboptimal dosage may provide an increased margin of safety, and may also reduce the cost of a drug necessary to achieve prophylaxis and therapy. In addition, a treatment utilizing a single combination dosage unit may provide increased convenience and may result in enhanced compliance. Other advantages of a composition, conjugate, or combination therapy may include higher stability towards degradation and metabolism, longer duration of action, and/or longer duration of action or effectiveness at particularly low doses.

In an aspect, the invention contemplates a composition, in particular a pharmaceutical composition, comprising an interferon and a modulator of ps20 polypeptides or polynucleotides. A pharmaceutical composition may optionally comprise a pharmaceutically acceptable carrier, excipient, or vehicle.

The invention also contemplates a pharmaceutical composition, comprising one or more interferon and one or more modulator of ps20 polypeptides or polynucleotides that provides beneficial effects relative to each compound alone. The beneficial effects provided by a composition of the invention can include enhanced therapeutic effects, in particular sustained therapeutic effects. A composition can have increased bioavailability (absorbed more rapidly and to a higher degree) or provide enhanced therapeutic effects.

The invention also provides a pharmaceutical composition for the treatment of a viral disease comprising a therapeutically effective amount of one or more interferon and one or more modulator of ps20 polypeptides or polynucleotides in a pharmaceutically acceptable carrier, excipient, or vehicle. In an embodiment, the composition is in a form such that administration to a subject results in reduction in viral load in the subject, in particular for a sustained period of time after cessation of treatment.

A method is provided for prolonging in a subject efficacy of an interferon therapy for treating a viral disease comprising administering to the subject receiving the therapy a therapeutically effective amount of a modulator of a ps20 polypeptide or ps20 polynucleotide, preferably a therapeutically effective amount to prolong the efficacy of the interferon therapy. The therapy and modulator may be administered simultaneously or sequentially, in any order and for any period of time. In an aspect, the modulator is administered (e.g. for a period of time or continuously) following completion of the interferon therapy.

In an aspect, the invention features a composition comprising one or more interferon and one or more modulator of ps20 polypeptides or polynucleotides, in dosages effective for inhibiting, reducing, or reversing a viral disease, in particular for a prolonged period following administration. In aspects of the invention, a therapeutic effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months following treatment. The period of time a therapeutic effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, or 2 weeks to 6 months, periodically or continuously.

In an aspect the invention provides a combination of an interferon and a modulator of ps20 polypeptides or polynucleotides in the treatment of conditions for which an interferon, in particular an alpha interferon, have been demonstrated to have a therapeutic effect.

A modulator of ps20 polypeptides or polynucleotides in a composition or combination treatment of the invention may be in a ratio selected to augment the activity of the interferon to provide a therapeutic effect. Combinations of interferons and modulators of ps20 polypeptides or polynucleotides in compositions of the invention may be selected to provide unexpectedly additive effects or greater than additive effects i.e. synergistic effects. Thus, the present invention includes combination treatments providing additive or synergistic activity, delivering an additive or synergistically effective amount, or an amount to provide a therapeutically effective amount of an interferon and a modulator of a ps20 polypeptide or ps20 polynucleotide, or a conjugate or composition of the invention. Pharmaceutical compositions suitable for use in the present invention include compositions wherein the active ingredients are contained in an effective amount, in particular a synergistically effective amount.

The invention also provides a pharmaceutical composition in separate containers and intended for simultaneous or sequential administration to a subject especially to provide therapeutic effects, comprising interferons and modulators of ps20 polypeptides or polynucleotides both optionally together with pharmaceutically acceptable carriers, excipients, or vehicles.

The invention further provides a conjugate comprising an interferon and a modulator of ps20 polypeptides or polynucleotides (e.g. antibodies specific for ps20 polypeptides or chimeric proteins).

The invention also provides a method of preparing a stable pharmaceutical composition comprising one or more interferons and modulators of ps20 polypeptides or polynucleotides. A method can comprise mixing one or more interferons and one or more modulators of ps20 polypeptides or polynucleotides and a pharmaceutically acceptable carrier, excipient, or vehicle, in particular, a pharmaceutically acceptable carrier, excipient, or vehicle effective to physically stabilize the interferons and modulators. After compositions have been prepared, they can be placed in an appropriate container and labeled for treatment of an indicated condition. For administration of a composition of the invention, such labeling would include amount, frequency, and method of administration.

The invention also contemplates the use of one or more interferons and modulators of ps20 polypeptides or polynucleotides, compositions, conjugates, or combination treatments of the invention for preventing, and/or ameliorating a viral disease, severity, disease symptoms, and/or periodicity of recurrence of a viral disease. The invention also relates to the prevention and treatment, in a subject, of viral diseases using one or more interferons and modulators of ps20 polypeptides or polynucleotides, compositions, combination treatments, and/or conjugates of the invention.

In a combination therapy to treat the diseases or conditions discussed herein, the interferon and modulator of ps20 polypeptide or polynucleotide can be administered simultaneously or at separate intervals. When administered simultaneously the interferon and modulator of ps20 polypeptide or polynucleotide can be incorporated into a single pharmaceutical composition, e.g., a pharmaceutical combination therapy composition. Alternatively, two or more separate compositions, i.e., one containing an interferon and the other(s) containing the modulator of ps20 polypeptide or polynucleotide can be administered simultaneously.

A pharmaceutical combination therapy composition can include therapeutically effective amounts of a modulator of ps20 polypeptide or polynucleotide, noted herein, and therapeutically effective amount of an interferon. The combined administration of an interferon and modulator of ps20 polypeptide or polynucleotide may require less of the generally-prescribed dose for any of agents when used alone and or may result in less frequent administration of either, both or all agents. These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and maybe formulated as sustained relief dosage forms and the like.

When separately administered, therapeutically effective amounts of compositions containing an interferon and modulator of ps20 polypeptide or polynucleotide are administered on a different schedule. One may be administered before the other as long as the time between the administrations falls within a therapeutically effective interval. A therapeutically effective interval is a period of time beginning when one of either (a) the interferon(s), or (b) the modulator(s) is(are) administered to a mammal and ending at the limit of the therapeutic effect in the treatment of the viral disease to be treated from the combination of (a) and (b). The methods of administration of the interferon and modulator of ps20 polypeptide or polynucleotide may vary. Thus, any of the agents may be administered rectally, topically, orally, sublingually, or parenterally.

In aspects of the invention, methods are provided for reducing or inhibiting a viral infection associated with a coronavirus or a coxsackievirus comprising directly or indirectly inhibiting a ps20 polypeptide, preferably inhibiting a ps20 polypeptide of SEQ ID NO. 2 or 3. In an embodiment of the invention, a method is provided for reducing or inhibiting a viral infection associated with a coronavirus or a coxsackievirus in a subject comprising administering an effective amount of an antagonist or inhibitor of a ps20 polypeptide. In particular, methods are provided for treating a patient suffering from or who may be susceptible to a viral disease associated with a coronavirus or a coxsackievirus comprising administering an effective amount of an antagonist of a ps20 polypeptide.

In an embodiment of the invention a method is provided for treating a patient who may be susceptible to a viral disease associated with a coronavirus or a coxsackievirus comprising administering therapeutically effective dosages of an antagonist or inhibitor identified in accordance with a method of the invention or described herein. Treatment with the antagonist or inhibitor is discontinued after ps20 polypeptide levels are within normal range, and before any adverse effects of administration of the inhibitor are observed.

A substance that reduces or inhibits a viral infection may be a molecule which interferes with the transcription and/or translation of a ps20 polypeptide, in particular a ps20 polypeptide of SEQ ID NO. 1. For example, the sequence of a nucleic acid molecule encoding a ps20 polypeptide or fragments thereof may be inverted relative to its normal presentation for transcription to produce an antisense nucleic acid molecule. An antisense nucleic acid molecule may be constructed using chemical synthesis and enzymatic ligation reactions using procedures known in the art.

Genes encoding a ps20 polypeptide can be turned off by transfecting a cell or tissue with vectors which express high levels of a desired ps20 polypeptide-encoding fragment. Such constructs can inundate cells with untranslatable sense or antisense sequences. Even in the absence of integration into the DNA, such vectors may continue to transcribe RNA molecules until all copies are disabled by endogenous nucleases. Vectors can be derived from retroviruses, adenovirus, herpes or vaccinia viruses, or from various bacterial plasmids, and used to deliver polynucleotides to a targeted cell population. Alternatively, antisense sequences may be introduced using lipid-based transfection technologies.

Modifications of gene expression can be obtained by designing antisense molecules, DNA, RNA or PNA, to the regulatory regions of a gene encoding a ps20 polypeptide, i.e., the promoters, enhancers, and introns. Preferably, oligonucleotides are derived from the transcription initiation site, for example, between about −10 and +10 regions of the leader sequence. Antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.). Methods well known to those skilled in the art may be used to construct recombinant vectors which will express antisense ps20 polynucleotides. (See, for example, the techniques described in Sambrook et al (supra) and Ausubel et al (supra)).

In an aspect, the invention provides a method of inhibiting expression of a gene encoding a ps20 polypeptide associated with a coronavirus or a coxsackievirus viral disease comprising the step of (i) providing a biological system in which expression of a gene encoding a ps20 polypeptide is to be inhibited; and (ii) contacting the system with an antisense molecule that hybridizes to a transcript encoding a ps20 polypeptide.

Antisense RNA transcripts of the present invention can have a base sequence complementary to part or all of a ps20 polynucleotide and modulate expression of ps20 polynucleotides. Antisense nucleic acids are generally single-stranded nucleic acids (DNA, RNA, modified DNA, or modified RNA) complementary to a portion of a target nucleic acid (e.g., an mRNA ps20 polynucleotide transcript) and are able to bind to the target to form a duplex. In aspects of the invention, an antisense is an oligonucleotide ranging from 10 to 50, in particular 15 to 35 nucleotides in length. Binding of the antisense molecule generally reduces or inhibits the function of the target ps20 polynucleotide. Reduction in expression of a ps20 polypeptide may be achieved by the administration of antisense nucleic acids or peptide nucleic acids comprising sequences complementary to those of the mRNA that encodes the ps20 polypeptide. [See the following for reviews of antisense technology and its applications: (Phillips, M. I. (ed.) Antisense Technology, Methods Enzymol., 313 and 314: 2000, and references mentioned therein; and Crooke, S. “Antisense Drug Technology: Principles, Strategies, and Applications” (1.sup.st Edition) Marcel Dekker; and references cited therein.

In aspects of the invention, a modulator of a ps20 polynucleotide is an interfering RNA (siRNA). RNA interference (RNAi) is a mechanism of post-transcriptional gene silencing modulated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a viral disease in a mammal by administering to the mammal a ps20 polynucleotide (e.g., dsRNA) that hybridizes under stringent conditions to a ps20 polynucleotide, and attenuates expression of the ps20 polynucleotide. RNAi is mediated by short interfering RNAs (siRNA), which generally comprises a double-stranded region approximately 19 nucleotides in length with 1-2 nucleotide 3′ overhangs on each strand, resulting in a total length of between approximately 21 and 23 nucleotides. dsRNA longer than about 30 nucleotides typically induces nonspecific mRNA degradation in mammalian cells. The presence of siRNA in mammalian cells results in sequence-specific gene silencing.

siRNAs may downregulate gene expression by transferring the siRNA into mammalian cells by methods such as transfection, electroporation, or microinjection, or when expressed in cells via any of a variety of plasmid-based approaches. [See the following for reviews of RNA interference using siRNA: Tuschl, Nat. Biotechnol. 20: 446-448, 2002; See also Yu, J., et al., Proc. Natl. Acad. Sci., 99: 6047-6052, 2002; Sui, et al., Proc. Natl. Acad. Sci USA. 99: 5515-5520, 2002; Paddison, et al., Genes and Dev. 16: 948-958, 2002; Brummelkamp, et al., Science 296: 550-553, 2002; Miyagashi, et al., Nat. Biotech. 20: 497-500, 2002; Paul, et al., Nat. Biotech. 20: 505-508, 2002]. A siRNA can comprise two individual nucleic acid strands or a single strand with a self-complementary region capable of forming a hairpin (stem-loop) structure. Variations in structure, length, number of mismatches, size of loop, identity of nucleotides in overhangs, etc., can be introduced into a siRNA to trigger effective siRNA gene silencing. In aspects of the invention, the siRNA targets exons rather than introns. In other aspects of the invention, a siRNA may comprise sequences complementary to regions within the 3′ portion of the target transcript.

siRNAs employed in aspects of the present invention include RNA strands containing two complementary elements that hybridize to one another to form a stem, a loop, and optionally an overhang, preferably a 3′ overhang. In particular aspects of the invention, the stem is approximately 19 base pairs long, the loop is about 1-20, more preferably about 4-10, and most preferably about 6-8 nucleotides long and/or the overhang is about 1-20, and more preferably are enzymatic RNA molecules that catalyze the specific cleavage of RNA. In certain aspects, the stem is at least 19 nucleotides in length and can be up to approximately 29 nucleotides in length. In particular aspects of the invention, the loops comprise 4 nucleotides or greater which are less likely to be subject to steric constraints than are shorter loops. An overhang can include a 5′ phosphate and a 3′ hydroxyl and may optionally comprise a plurality of U residues, for example about 1 and 5 U residues. Classical siRNAs trigger degradation of mRNAs to which they are targeted to thereby reduce the rate of protein synthesis. In addition to classical siRNAs, certain siRNAs bind to the 3′ UTR of a template transcript and can inhibit expression of a protein encoded by the template transcript by reducing translation of the transcript rather than decreasing its stability. These RNAs are referred to as microRNAs (mRNAs) which can be between about 20 and 26 nucleotides in length, e.g., 22 nucleotides in length. mRNAs may be derived from larger precursors known as small temporal RNAs (stRNAs) or mRNA precursors, which are typically approximately 70 nucleotides long with an approximately 4-15 nucleotide loop. (For example, see Grishok, et al., Cell 106: 23-24, 2001; Hutvagner, et al., Science 293: 834-838, 2001; Ketting, et al., Genes Dev., 15: 2654-2659, 2001). MicroRNAs have been found to block translation of target transcripts containing target sites in mammalian cells (Zeng, et al., Molecular Cell 9: 1-20, 2002).

In an embodiment, the invention provides a method of inhibiting expression of a gene encoding a ps20 polypeptide associated with a coronavirus or a coxsackievirus viral disease comprising the step of (i) providing a biological system in which expression of a gene encoding a ps20 polypeptide is to be inhibited; and (ii) contacting the system with a siRNA targeted to a transcript encoding ps20 polypeptide. In embodiments of the invention, the biological system comprises a cell (e.g., CD4 T cells), and the contacting step comprises expressing the siRNA in the cell. In other embodiments, the biological system comprises a subject, e.g., a mammalian subject such as a mouse or human, infected with a coronavirus or a coxsackievirus, and the contacting step comprises administering the siRNA to the subject or comprises expressing the siRNA in the subject. According to certain embodiments of the invention the siRNA is expressed inducibly and/or in a cell-type or tissue specific manner.

A variety of RNA molecules containing duplex structures may be employed to mediate silencing of ps20 polynucleotides through various mechanisms. Any such RNA, one portion of which binds to a target transcript and reduces its expression, whether by triggering degradation, by inhibiting translation, or by other means, is considered to be an siRNA, and any structure that generates such an siRNA is useful in the practice of the present invention.

In aspects of the invention, hairpin structures that mimic siRNAs and mRNA precursors are employed. These structures may be processed intracellularly into molecules capable of reducing or inhibiting expression of target ps20 transcripts (for example, see McManus, et al., RNA 8: 842-850, 2002). These structures which are based on classical siRNAs comprising two RNA strands forming a 19 base pair duplex structure are classified as class I or class II hairpins. Class I hairpins incorporate a loop at the 5′ or 3′ end of an antisense siRNA strand (i.e., the strand complementary to the target transcript whose inhibition is desired) but are otherwise identical to classical siRNAs. Class II hairpins resemble mRNA precursors and comprise a 19 nucleotide duplex region and a loop at either the 3′ or 5′ end of the antisense strand of the duplex in addition to one or more nucleotide mismatches in the stem. Hairpins are processed intracellularly into small RNA duplex structures capable of mediating silencing.

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a ps20 polypeptide. Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

In aspects of the invention, a composition is provided for treating a patient suffering from, or who may be susceptible to a coronavirus or a coxsackievirus viral disease, comprising a therapeutically effective amount of an antagonist or inhibitor of a ps20 polypeptide, or substance selected in accordance with the methods of the invention, including binding agents (e.g. antibodies, antisense or siRNAs) and a carrier, diluent, or excipient.

A composition of the invention contains a therapeutically effective dose of an inhibitor of a ps20 polypeptide, for example, an amount sufficient to lower levels of ps20 polypeptide to normal levels is about 1 to 1000, 1 to 500, 1 to 250, 1 to 200, 1 to 150, 1 to 100 or 1 to 50 μg/kg/day. A method of the invention for treating and/or preventing a viral infection in particular a viral infection associated with a coronavirus or a coxsackievirus, may involve a series of administrations of the composition. Such a series may take place over a period of 7 to about 21 days and one or more series may be administered. The composition may be administered initially at the low end of the dosage range and the dose will be increased incrementally over a preselected time course.

An antagonist or inhibitor of a ps20 polypeptide, or a substance identified in accordance with a method of the invention may be administered by gene therapy techniques using genetically modified cells or by directly introducing genes encoding the inhibitors or stimulators into cells (e.g., T cells) in vivo. Cells may be transformed or transfected with a recombinant vector (e.g. retroviral vectors, adenoviral vectors and DNA virus vectors). Genes encoding inhibitors or stimulators, or substances may be introduced into cells of a subject in vivo using physical techniques such as microinjection and electroporation or chemical methods such as coprecipitation and incorporation of DNA into liposomes. Antisense molecules may also be introduced in vivo using these conventional methods.

One or more ps20 polypeptides or ps20 polynucleotides may be targets for immunotherapy. Immunotherapeutic methods include the use of antibody therapy, in vivo vaccines, and ex vivo immunotherapy approaches. In one aspect, the invention provides one or more antibodies specific for one or more ps20 polypeptides that may be used to treat a viral disease associated with a coronavirus or a coxsackievirus.

Thus, the invention provides a method of treating a patient susceptible to, or having a viral disease, in particular a viral disease associated with a coronavirus or a coxsackievirus, comprising administering to the patient an effective amount of an antibody that binds specifically to one or more ps20 polypeptide.

The invention encompasses administration of antibodies or fragments thereof that immunospecifically bind to and antagonize ps20 polypeptides. In an embodiment, the antibody binds to a WAP domain of a ps20 polypeptide and, preferably, also antagonizes ps20 polypeptides. In other embodiments, the antibodies inhibit or reduce permissiveness of CD4 T cells or other cells that are permissive for virus infection, rescue or destroy CD4 T cells that are susceptible to viral infection. In another embodiment, the antibody binds to a ps20 polypeptide or domain or fragment thereof, preferably with a K_{off} of less than 3 ×10⁻³ to 10×10⁻³.

One or more ps20 polypeptide antibodies may also be used in a method for selectively inhibiting or killing CD4 T cells (e.g. CD45RO+/CD28+/CD5T cells) or other cells that are targets of virus infection, secreting one or more ps20 polypeptide, in particular in a subject with a viral disease associated with a coronavirus or a coxsackievirus infection, comprising reacting one or more ps20 polypeptide antibody immunoconjugate or immunotoxin with the cell in an amount sufficient to inhibit or kill the cell. By way of example, unconjugated antibodies to ps20 polypeptides may be introduced into a patient such that the antibodies bind to ps20 polypeptides expressed by cells. In addition to unconjugated antibodies to ps20 polypeptides, one or more ps20 polypeptide antibodies conjugated to therapeutic agents (e.g. immunoconjugates) may also be used therapeutically to deliver the agent directly to one or more ps20 polypeptide expressing cells and thereby destroying the cells. Examples of such agents include abrin, ricin A, Pseudomonas exotoxin, or diphtheria toxin.

In the practice of a method of the invention, ps20 polypeptide antibodies capable of inhibiting or killing cells expressing ps20 polypeptides are administered in a therapeutically effective amount to patients with a viral disease. The invention may provide a specific and effective treatment for a viral disease. The antibody therapy methods of the invention may be combined with other therapies including interferons.

ps20 polypeptide antibodies useful in treating a viral disease include those that are capable of initiating a potent immune response against the disease and those that are capable of directing cytotoxicity. In this regard, ps20 polypeptide antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

ps20 polypeptide antibodies that exert a direct biological effect on cells expressing ps20 polypeptides may also be useful in the practice of the invention. Such antibodies may not require the complete immunoglobulin to exert the effect. The mechanism by which a particular antibody exerts an effect may be evaluated using any number of in vitro assays designed to determine ADCC, antibody-dependent macrophage-mediated cytotoxicity (ADMMC), complement-mediated cell lysis, and others known in the art.

The methods of the invention contemplate the administration of single ps20 polypeptide antibodies as well as combinations, or “cocktails”, of different individual antibodies such as those recognizing different epitopes of other markers. Such cocktails may have certain advantages inasmuch as they contain antibodies that bind to different epitopes of ps20 polypeptides. Such antibodies in combination may exhibit synergistic therapeutic effects. In addition, the administration of one or more ps20 polypeptide specific antibodies may be combined with other therapeutic agents, including but not limited to antibiotics and biological factors such as interferons, in particular Type I interferons, more particularly an IFN-alpha. ps20 polypeptide specific antibodies may be administered in their “naked” or unconjugated form, or may have therapeutic agents conjugated to them.

ps20 polypeptide specific antibodies used in the present invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington: The Science and Practice of Pharmacy. (21st Edition, Popovich, N (eds), Advanced Concepts Institute, University of the Sciences in Philadelphia, Philadelphia, Pa. 2005).

One or more ps20 polypeptide specific antibody formulations may be administered via any route capable of delivering the antibodies to the disease site. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection. Treatment will generally involve the repeated administration of the antibody preparation via an acceptable route of administration such as intravenous injection (IV), at an effective dose.

Dosages of antibodies will depend upon various factors generally appreciated by those of skill in the art, including the type of disease and the severity, stage of the disease, the binding affinity and half life of the antibodies used, the degree of ps20 polypeptide expression in the patient, the extent of ps20 polypeptides, the desired steady-state antibody concentration level, frequency of treatment, and the influence of any therapeutic agents used in combination with the treatment method of the invention. Daily doses may range from about 0.01 to 500 mg/kg, 0.1 to 200 mg/kg, or 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more ps20 polypeptide antibodies is also possible and may have advantages in certain situations.

Patients may be evaluated for serum or tissue ps20 polypeptides, ps20 polynucleotides and/or interferons in order to assist in the determination of the most effective dosing regimen and related factors or to select a patient population for treatment. Conventional assay methods may be used for quantitating circulating ps20 polypeptide levels in patients prior to treatment. Such assays may also be used for monitoring throughout therapy, and may be useful to gauge therapeutic success in combination with evaluating other parameters.

The invention further provides vaccines formulated to contain one or more ps20 polypeptide or fragment thereof. In an embodiment, the invention provides a method of vaccinating an individual against one or more ps20 polypeptide comprising the step of inoculating the individual with the marker or fragment thereof that lacks activity, wherein the inoculation elicits an immune response in the individual thereby vaccinating the individual against the marker.

Viral gene delivery systems may be used to deliver one or more ps20 polynucleotides or ps20 polypeptides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding one or more ps20 polypeptide or fragment thereof introduced into the patient (e.g., intramuscularly) to induce a response.

Anti-idiotypic ps20 polypeptide specific antibodies can also be used in therapy as a vaccine for inducing an immune response. The generation of anti-idiotypic antibodies is well known in the art and can readily be adapted to generate anti-idiotypic ps20 polypeptide specific antibodies that mimic an epitope on one or more ps20 polypeptides (see, for example, Wagner et al., 1997, Hybridoma 16: 33-40; Foon et al., 1995, J Clin Invest 96: 334-342). Such an antibody can be used in anti-idiotypic therapy as presently practiced with other anti-idiotypic antibodies directed against antigens associated with disease.

Genetic immunization methods may be utilized to generate prophylactic or therapeutic humoral and cellular immune responses. One or more DNA molecules encoding ps20 polypeptides, constructs comprising DNA encoding one or more ps20 markers/immunogens and appropriate regulatory sequences may be injected directly into muscle or skin of an individual, such that the cells of the muscle or skin take-up the construct and express the encoded ps20 markers/immunogens. The ps20 markers/immunogens may be expressed as cell surface proteins or be secreted. Expression of one or more ps20 markers results in the generation of prophylactic or therapeutic humoral and cellular immunity against a viral disease. Various prophylactic and therapeutic genetic immunization techniques known in the art may be used.

In another aspect, the invention provides methods for selectively inhibiting expression of ps20 polypeptide by reacting any one or a combination of the immunoconjugates of the invention with the cells secreting ps20 polypeptides in an amount sufficient to inhibit ps20 activity.

Methods well known to those skilled in the art may be used to construct recombinant vectors that will express antisense polynucleotides for ps20 polypeptides. (See, for example, the techniques described in Sambrook et al (supra) and Ausubel et al (supra)).

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

The therapeutic activity of compositions and agents/compounds identified using a method of the invention and may be evaluated in vivo using suitable animal models, for example the animal models described in the examples.

The following non-limiting examples are illustrative of the present invention:

### Example 1

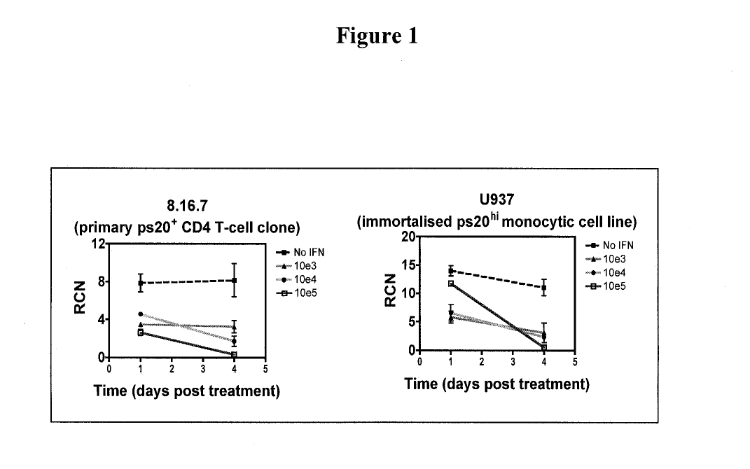

Given the central importance of interferon alpha as an innate antiviral factor involved in host immunity against both HIV and influenza virus infections, experiments were conducted to examine whether the antiviral effect of IFN alpha involved interfering with the ps20 pathway. In vitro experiments were conducted in two cell populations that have previously been shown to be highly susceptible to HIV and to be ps20+: a ps20+ CD4 T-cell clone, 8.16.7 and the ps20+ immortalized cell line of monocytic lineage-U937. The graphs in FIG. 1 show that exposing each of the two cell populations to recombinant interferon alpha significantly reduces endogenous ps20 mRNA expression measured by qRT-PCR relative to a house-keeping gene. Other data (not shown) demonstrate that there is concomitant induction of cellular HIV resistance in these cells.

### Example 2

Assaying ps20

Rapid functional assay: A critical pre-requisite for drug development is the need for a rapid assay for biological activity. The following are two such assays:

(a) Regulation of cell-adhesion molecules: It would appear that reconfiguration of cell surface molecules involved in cell-cell adhesion occurs upstream of the ps20/virus effect as indicated by down-regulation of the CD54 integrin by anti-ps20 antibody concomitant with induction of cellular HIV resistance. Therefore CD54 integrin regulation determined by FACS is an appropriate assay.

(b)Single-cycle HIV infection: Stable ps20 expression has been engineered in Jurkat cells and populations have been identified that faithfully recapitulate the endogenous ps20 effect. The expression of an indicator gene (luciferase) under the control of an HIV-1 promoter is being engineered in these cells. Monitoring luciferase expression in the ps20^{hi} Jurkats in the first 24 hours of HIV challenge would be a rapid second assay for ps20 function.

### Example 3

None of the reported models of SARS produce lung pathology similar to that seen in humans. Fish et al. [De Albuquerque et al, J Virol 2006, 80:10382-94]. demonstrated that intranasal MI-IV-1 infection of inbred strains of mice produces a lethal SARS-like disease in A/J mice with features similar to those found in human patients, whereas other mouse strains including C57BL/6J mice fully recover. Fish went on to further define differences in the type I IFN response in resistant and susceptible mice and confirmed a role for IFN in treatment for the viral disease. The extent of IFN expression induced on virus infection was found to directly correlate with susceptibility to virus infection (Baig, E and E. N. Fish, 2008, Antiviral Therapy 13: 409-422). A/J mice that are highly susceptible to intranasal challenge with MHV-1 and develop an aggressive respiratory infection, exhibit lower levels of inducible IFN-α and IFN-β gene expression in affected lungs compared with resistant C57B1/6 mice. These studies were extended to examine MHV-1 inducible IFN gene expression in the draining mediastinal lymph nodes (mLN) of MHV-1 infected A/J and C57B1/6 mice. The mLN were chosen since a subpopulation of dendritic cells, plasmacytoid dendritic cells (pDC), which reside in the LN are regarded as the principle IFN producing cells. MHV-1-inducible IFN gene expression levels in mLN tissue harvested from C57B1/6 mice were consistently higher at all time points examined compared with IFN gene expression levels in mLN tissue from A/J mice.

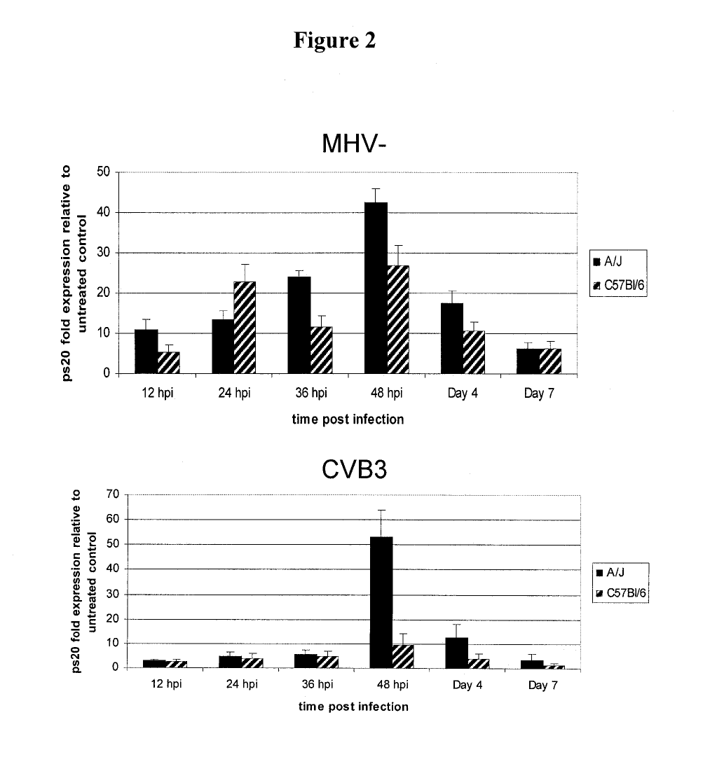

Experiments were conducted to examine whether levels of ps20 gene expression distinguished between susceptible and resistant mice, specifically examining the target tissue of MHV-1 infection, namely the lungs. The data in the upper panel of FIG. 2 demonstrates that at 48 hours post-infection, when A/J mice exhibit severe progressive pulmonary disease, gene expression levels for ps20 are significantly higher than expression levels expressed in the C57BL/6 mice.

Viewed altogether, the data identify a direct correlation between susceptibility to lung respiratory MHV-1 infection and ps20 levels. Moreover, the data are consistent with an inverse correlation between IFN gene expression levels and ps20 levels, i.e. that a robust IFN response is inversely correlated with lower ps20 levels in the same tissues.

### Example 4

Coxsackievirus group B enteroviruses, and specifically CVB3, are among the primary agents of virus induced myocarditis. Different strains of mice are differentially susceptible to CVB3 induced disease [see Deonarain, R. et al., Circulation 2004, 110:3540-3]. IFN-β has been found to be important in protection from CVB3 inducible myocarditis. Therefore, CVB3 inducible type I IFN gene expression levels were examined in susceptible A/J and resistant C57Bl/6 mice to determine whether the extent of an IFN response to viral challenge correlates with disease susceptibility. C57Bl/6 mice exhibited higher levels of gene expression for all detected type I IFNs in heart tissue compared to A/J mice ((Baig, E and E. N. Fish, 2008, Antiviral Therapy 13: 409-422).

Experiments were also conducted to examine whether levels of ps20 gene expression distinguished between susceptible and resistant mice, specifically examining the target tissue of CVB3 infection, namely the heart. The data in the lower panel of FIG. 2 demonstrate that whereas ps20 gene expression levels in the infected and resistant C57BL/6 mice remained low throughout the course of disease, at 48 hours post-infection ps20 gene expression had increased >50-fold above basal expression in the hearts of infected and susceptible A/J mice.

Viewed altogether, the data also identify a direct correlation between susceptibility to CVB3 infection and ps20 levels. Moreover, the data are consistent with an inverse correlation between IFN gene expression levels and ps20 levels, i.e. that a robust IFN response is inversely correlated with lower ps20 levels in the same tissues.

### Example 4

### Influenza Virus H5N1 Infection of Human Lung Tissue

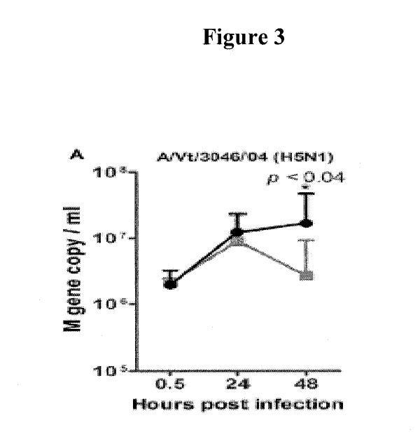

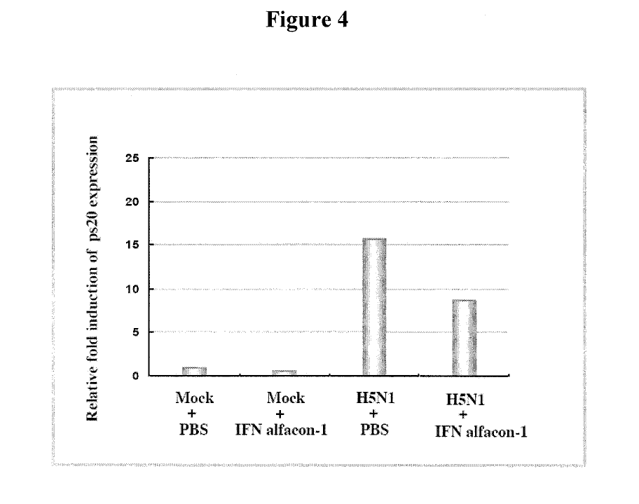

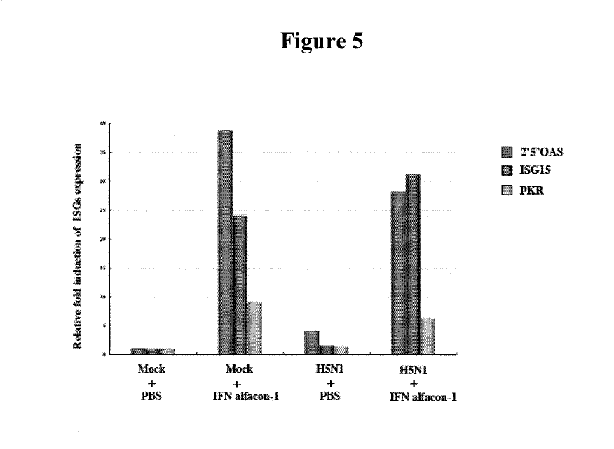

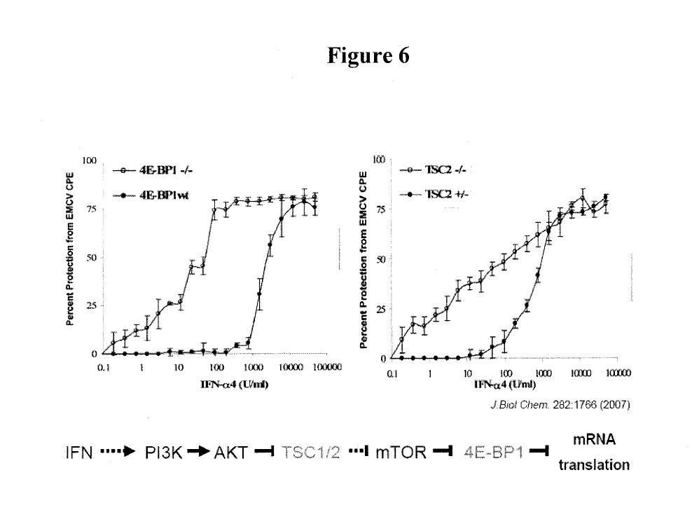

Primary human lung tissue segments (5mm cube) were isolated from human non-tumor lung tissue obtained from patients undergoing lung resection in Graham Hospital, Hong Kong. Tissue was either left untreated or treated with IFN alfacon-1 at 10,000U/ml at −30 mins relative to infection with influenza A/VN/3046/04 (H5N1) virus, titer 10e 8.33TCID50/mL. At 0.5, 24 and 48 hrs post infection, tissue was processed for examination of viral M gene copy number, and at 18 hr post-infection for ps20 gene expression and the IFN-inducible genes associated with an antiviral response, namely 2′5′ -oligoadenylate synthetase (2′5′-OAS), PKR and ISG15. The data in FIG. 3 demonstrate that these human lung explants are infected with influenza virus H5N1, since M gene copy number increases over the time course examined. Notably, IFN alfacon-1 treatment results in a reduction in viral replication that is significant at the 48 hr time point. Moreover, at 18hr post-infection with influenza virus H5N1 there is an increase in ps20 gene expression above uninfected tissue (FIG. 4) and that IFN alfacon-1 treatment reduces this ps20 gene expression (FIG. 4). FIG. 5 shows that treatment of human lung explants with IFN alfacon-1 results in an upregulation of the 3 IFN-inducible antiviral proteins 2′5′-OAS, PKR and ISG15, whereas infection with influenza virus H5N1 fails to increase the expression of these genes, suggestive of a failed IFN response. However, IFN treatment results in an increase in these antiviral proteins in H5N1-infected lung tissue, from which it is concluded that these IFN-inducible antiviral proteins likely mediate the reduction in M gene copy number (FIG. 6).

### Example 5

ps20 Gene Expression in 4E-BP1 Null Mice

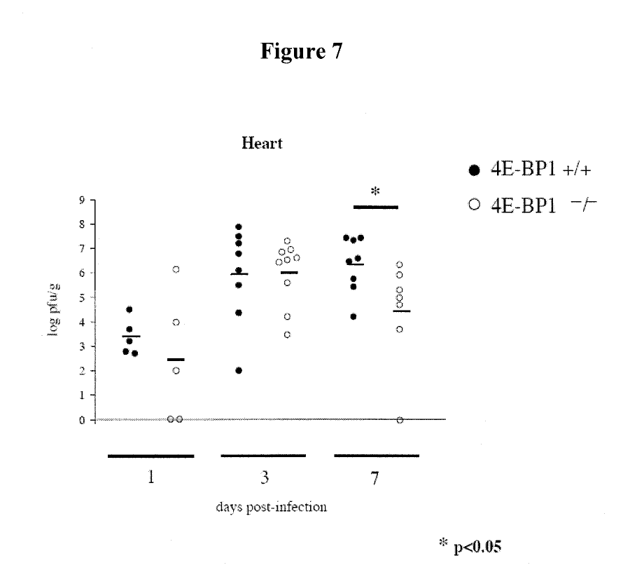

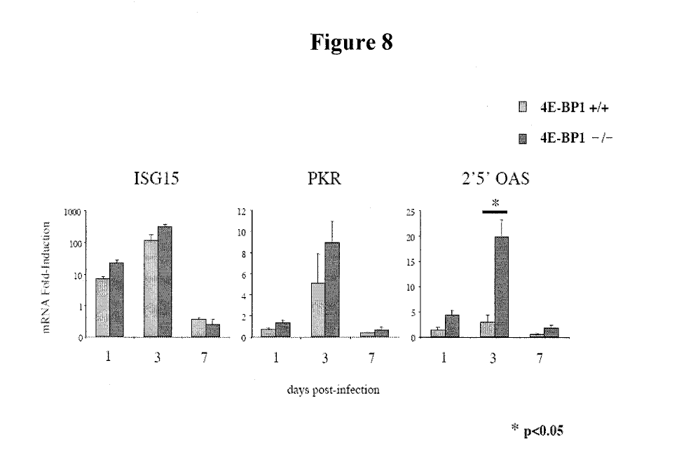

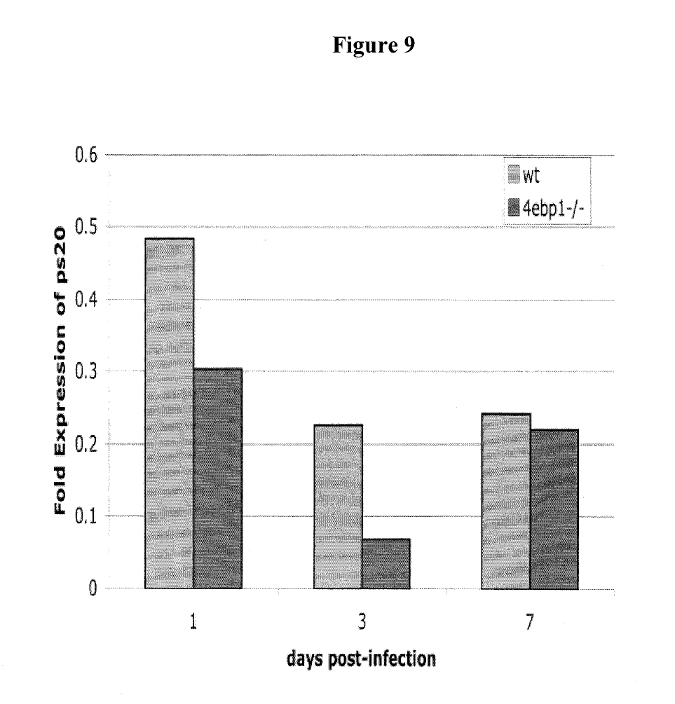

Distinct from the ability of IFNs-αβ to invoke signaling cascades that result in transcriptional activation, there is evidence for IFN-mediated effects on mRNA translation. Specifically, it has been shown that IFN-α induces phosphorylation/inactivation of the 4E-BP 1 repressor of mRNA. Cells that are null for 4E-BP 1 are more resistant to virus infection and more sensitive to the antiviral effects of IFN (Lekmine, F., Uddin, S., Sassano, A., Parmar, S., Majchrzak, B., Hay, N., Fish, E. N., Platanias, L. C. (2003) J. Biol. Chem. 278: 27772-27780) (FIG. 5). In vivo, evidence shows that 4E-BP1 null mice infected with CVB3 are more resistant to infection than their wildtype counterparts (FIG. 7). The data in FIG. 8 indicate that the difference in susceptibility to infection may be a consequence of de-repression of mRNA translation for the antiviral effectors 2′5′-OAS, PKR and ISG15: evidence has been provided for increased expression of these antiviral effectors in the hearts of the 4E-BP1 null mice. Additionally, ps20 gene expression levels are lower in the hearts of 4E-BP 1 null mice than their wildtype counterparts at early time points post-infection (days 1, 3), correlating with reduced infectivity and enhanced expression of antiviral effectors (FIG. 9); i.e. the data are consistent with ps20 gene expression correlating directly with viral infectivity.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### Sequence Listing

| SEQ ID NO.: 1 | |
|---|---|
| NM_021197 1396 bp mRNA linear | |
| Homo sapiens WAP four-disulfide core domain 1 (WFDC1), mRNA | |
| 1 | agccaccatc gaggaagggg catgtgctgg acgcggacac atgatccgag ggaccctgct |
| 61 | gggtggaact aagaaagtcc agcagactgt gcatgctcct gtccccactc acaggcccac |
| 121 | gcagcgaggg gggcccctct tctgtgtgcg tctggaaggt cgctgcccag ggaggaaatg |
| 181 | cctttaaccg gcgtggggcc gggcagctgc aggaggcaga tcatccgggc tctgtgcctc |
| 241 | ttgctacttc tcctccacgc cggctctgcc aagaatatct ggaaacgggc attgcctgcg |
| 301 | aggctggccg agaaatcccg tgccgaggag gcgggcgcgc ccggcggccc ccggcagccc |
| 361 | cgagcagacc gctgcccgcc gcctccgcgg acgctgcccc ccggcgcctg ccaggccgcg |
| 421 | cgctgtcagg cggactccga gtgcccgcgg caccggcgct gctgctacaa cggatgcgcc |
| 481 | tacgcctgcc tagaagctgt gccgcccccg ccagtcttag actggctggt gcagccgaaa |
| 541 | cctcgatggc ttggtggcaa tggctggctc ctggatggcc ctgaggaggt gttacaagca |
| 601 | gaggcgtgca gcaccacgga ggatggggcc gaacccctgc tctgtccctc gggctatgag |
| 661 | tgccacatcc tgagcccagg tgacgtggcc gaaggtatcc ccaaccgtgg gcagtgcgtc |
| 721 | aagcagcgcc ggcaagcaga tgggcgaatc ctacgacaca aactttacaa agaatatcca |
| 781 | gaaggtgact caaagaatgt ggcagaacct ggaaggggac aacagaagca ctttcagtaa |
| 841 | agcaacggca agcagctagg ttgcaagaac attcctctac tttctgctaa gccttggaaa |
| 901 | cagttgggaa aagtagtttg accctcacag ttcacattca gctcagcaga gcaagacccc |
| 961 | agagatgctt agagacagga cacctggccc tcaaacccag tttggcccag cctggttggg |
| 1021 | tgactttgtg ggagccactt aacagctctg ggtccctgtt ttaccatcct gggagcaagg |
| 1081 | ccctgcagct ccacgagacc tttaccccgg gaagaagccg ccgcccatga aagcatttct |
| 1141 | gaagcccctt tctaagacaa ggctcagcat cttgatattt ttgacagatt cctcccaagt |
| 1201 | ctggctctgg gaggtatgta cccatctcaa atgttcccaa gataaattca tccttcagga |
| 1261 | aatggaaatg aacttgctta ctaatgtgtg attcctagtt gtagccaccg gatgtgctga |
| 1321 | ggcctaaatg ttagcaggtg ggaggaggcc acagaacaat aaaaacaacc aaataagaaa |
| 1381 | aaaaaaaaaa aaaaaa |
| SEQ ID NO. 2 | |
| NP_067020 220 aa linear | |
| WAP four-disulfide core domain 1 precursor [Homo sapiens]. | |
| 1 | mpltgvgpgs crrqiiralc llllllhags akniwkralp arlaeksrae eagapggprq |
| 61 | pradrcpppp rtlppgacqa arcqadsecp rhrrccyngc ayacleavpp ppvldwlvqp |
| 121 | kprwlggngw lldgpeevlq aeacsttedg aepllcpsgy echilspgdv aegipnrgqc |
| 181 | vkqrrqadgr ilrhklykey pegdsknvae pgrgqqkhfq |
| SEQ ID NO. 3 | |
| EAW95486 220 aa linear | |
| WAP four-disulfide core domain 1, isoform CRA_a [Homo sapiens] | |
| EAW95487 220 aa linear | |
| WAP four-disulfide core domain 1, isoform CRA_a [Homo sapiens] | |
| AAG16647 220 aa | |
| prostate stromal protein ps20 [Homo sapiens]. | |
| Q9HC57 220 aa linear | |
| WAP four-disulfide core domain protein 1 precursor (Prostate stromal | |
| protein ps20) (ps20 growth inhibitor). | |
| 1 | mpltgvgpgs crrqiiralc llllllhags akniwkralp arlaeksrae eagapggprq |
| 61 | pradrcpppp rtlppgacqa arcqadsecp rhrrccyngc ayacleavpp ppvldwlvqp |
| 121 | kprwlggngw lldgpeevlq aeacsttedg aepllcpsgy echilspgdv aegipnrgqc |
| 181 | vkqrrqadgr ilrhklykey pegdsknvae pgrgqqrhfq |

1. A method for diagnosing a viral disease in a subject comprising:
(a) assaying a biological sample from a subject for one or more ps20 polypeptides or ps20 polynucleotides and interferons or polynucleotides encoding interferons; and
(b) comparing the amount of ps20 polypeptides or ps20 polynucleotides and interferons or polynucleotides encoding interferons assayed to a predetermined standard, where detection of amounts of ps20 polypeptides or ps20 polynucleotides and interferons or polynucleotides encoding interferons that differ significantly from the standard indicates a viral disease or susceptibility or predisposition to a viral disease.

2. A method as claimed in claim 1 wherein the amount of ps20 polypeptides or ps20 polynucleotides assayed is greater than that of a standard and the amount of interferons or polynucleotides encoding interferons is lower than that of a standard.

3. A method as claimed in claim 1 comprising:
(a) contacting the biological sample with a binding agent that specifically binds to the ps20 polypeptides and a binding agent that specifically binds to the interferons; and
(b) detecting in the sample amounts of ps20 polypeptides and interferons that bind to the binding agents, relative to a predetermined standard or cut-off value, and therefrom determining the presence or absence of the viral disease in the subject.

4. A method as claimed in claim 3 wherein the binding agent is an antibody.

5. A method as claimed in claim 1 comprising detecting ps20 polynucleotides and polynucleotides encoding interferons in the sample and relating the detected amount to the presence of the viral disease.

6. A method as claimed in claim 5 wherein the polynucleotides are detected by
(a) contacting the sample with oligonucleotides that hybridize to ps20 polynucleotides and oligonucleotides that hybridize to polynucleotides encoding interferons; and
(b) detecting in the sample levels of ps20 polynucleotides and polynucleotides that encode interferons that hybridize to the oligonucleotides and comparing the levels to a predetermined standard or cut-off value, and therefrom determining the presence or absence of a viral disease in the subject.

7. A method as claimed in claim 5 wherein the polynucleotide detected is mRNA.

8. A method as claimed in claim 7 wherein the mRNA is detected using an amplification reaction.

9. A method as claimed in claim 8 wherein the amplification reaction is a polymerase chain reaction employing oligonucleotide primers that hybridize to the polynucleotides, or complements of such polynucleotides.

10. A method as claimed in claim 7 wherein the mRNA is detected using a hybridization technique employing oligonucleotide probes that hybridize to the polynucleotides or complements of such polynucleotides.

11. A method as claimed in claim 5 wherein the presence of higher levels of polynucleotides encoding ps20 polypeptides and lower levels of polynucleotides encoding interferons in the sample compared to a standard or control is indicative of a viral disease or prognosis.

12. A method according to claim 1 for monitoring the progression of a viral disease in a subject, the method further comprising: (c) repeating steps (a) and (b) at a subsequent point in time; and comparing levels detected at each time point, and thereby monitoring the progression of the viral disease.

13. A method as claimed in claim 1 wherein the viral disease is associated with a coxsackievirus or a coronavirus.

14. (canceled)

15. A method for treating a viral disease in a subject, comprising administering to the subject a combination of a therapeutically effective amount of at least one interferon and a therapeutically effective amount of at least one antagonist of a ps20 polypeptide or a ps20 polynucleotide.

16. A method as claimed in claim 15 wherein the viral disease is caused by influenza or associated with a coxsackievirus or a coronavirus.

17. A method as claimed in claim 15 wherein the ps20 antagonist is a purified antibody that immunospecifically binds to a ps20 polypeptide including fragments thereof.

18. A method as claimed in claim 15 wherein the antagonist is a humanized antibody, an antisense ps20 polynucleotide or an interfering RNA (siRNA) targeted to a transcript encoding a ps20 polypeptide.

19. (canceled)

20. A composition for treating a viral disease comprising an interferon and an antagonist of a ps20 polypeptide or a ps20 polynucleotide.

21. A composition as claimed in claim 20 wherein the antagonist is an antisense ps20 polynucleotide, an interfering RNA (siRNA) targeted to a transcript encoding a ps20 polypeptide, or a purified antibody that immunospecifically binds to a ps20 polypeptide including fragments thereof.

22. (canceled)

23. A kit for carrying out a method as claimed in claim 1.
